(19) United States
(12) Patent Application Publication
(10) Pub. No.: US 2021/0254092 A1
(43) Pub. Date: Aug. 19, 2021

(54) Methods of Producing Insect Pheromones

(71) Applicants: Jan Christer Lofstedt, Lund (SE); Per Fredrik Hofvander, Bjarred (SE); Honglei Wang, Lund (SE); Baojian Ding, Lund (SE); Yihan Xia, Lund (SE); Carin Ingegerd Jarl-Sunesson, Lund (SE); Board of Regents of the University of Nebraska, Lincoln, NE (US); ISCA Technologies, Inc., Riverside, CA (US)

(72) Inventors: Jan Christer Lofstedt, Lund (SE); Per Fredrik Hofvander, Bjarred (SE); Baojian Ding, Lund (SE); Yihan Xia, Lund (SE); Honglei Wang, Lund (SE); Carin Ingegerd Jarl-Sunesson, Lund (SE); Edgar Benjamin Cahoon, Lincoln, NE (US); Agenor Mafra-Neto, Riverside, CA (US)

(21) Appl. No.: 17/169,444

(22) Filed: Feb. 6, 2021

Related U.S. Application Data
(60) Provisional application No. 62/971,692, filed on Feb. 7, 2020.

(51) Int. Cl. C12N 15/82

(52) U.S. Cl. CPC C12N 15/8286; C12N 15/8247

(57) ABSTRACT

The present disclosure relates to methods of producing insect pheromone precursors and genetically modified plants capable of producing insect pheromone precursors. The genetically modified plants include a heterologous gene encoding at least one silencing suppressor protein and at least one enzyme selected from the group consisting of a fatty acyl desaturase, a fatty acyl elongase, a fatty acyl reductase, and an acyl-CoA oxidase.

### CROSS-REFERENCE TO RELATED APPLICATIONS

This application claims the benefit of U.S. Provisional Application No. 62/971,692, filed on Feb. 7, 2020, titled METHODS OF PRODUCING INSECT PHEROMONES, the teachings of which are expressly incorporated by reference.

### STATEMENT RE: FEDERALLY SPONSORED RESEARCH/DEVELOPMENT

This invention was made with government support under DE-SC0001295 awarded by the United States Department of Energy. The government has certain rights in the invention.

### BACKGROUND

### Field of the Invention

The present disclosure relates generally to the production of insect pheromones in plants, and more particularly to the use of P19 to increase the production of insect pheromones in plants.

### Description of the Prior Art

Moths are well known as a pest to crops. In this regard, the female moths lay eggs on the crops and the hatched larvae will feed on the crops, causing serious damage. Moths heavily rely on sex pheromones to communicate between males and females for mating. Pheromones are molecules used for communication between living organisms. In particular, female moths emit species-specific sex pheromone component blends to attract males of the same species over long distances. There are more than 160,000 species described in the order Lepidopteran (moths and butterflies). They are among the most damaging pests of food and fiber crops, capable of quickly adapting and evolving resistance to insecticides. Of the downsides known with conventional insecticides for pest control is that they do not discriminate between pest and other non-target insects and can, in many cases, be harmful to other organisms, including humans, and detrimental to plants that are dependent on beneficial insects for pollination. Using pheromones for pest control (such as mass trapping and mating disruption) has become an environmentally friendly alternative because the pheromones are non-toxic, they have no adverse effects on non-target organisms, they do not kill parasitoids or other beneficial insects, and the risks of resistance being developed in the pest is small. Even in terms of profit and reduction in damage, pheromones often compare favorably to the use of insecticides. The global market for pheromone-based control products is currently estimated to more than $200 million. However, current standard approaches to pheromone synthesis either require the use of hazardous chemicals or may result in the production of hazardous waste by-products, and it is most difficult to modify the double bonds for production of unsaturated pheromone precursors. The problems inherent to synthetic pheromone production may be overcome by developing an innovative green chemistry alternative, minimizing hazards. Along those lines, it has been previously disclosed in International Patent Publication No. WO 2015/171057, the use of plants to produce insect pheromone precursors, the entirety of which is incorporated herein by reference.

Indeed, a majority of the identified moth sex pheromone components consists of fatty acid derivatives, which are biosynthesized in species-specific pathways involving successive enzyme activities. Among the enzymes, fatty acyl desaturases (FADs) play an important role in producing the great diversity of sex pheromones between species, introducing double bonds in specific positions of the fatty acyl chain to form the pheromone skeletons. By benefiting from current development of biotechnology and bioengineering, and the functionally characterized pheromone biosynthetic gene toolbox, such as FADs, it has now become possible to synthesize customized pheromones in transformed organisms efficiently. The present disclosure discusses the use of several plant platforms (tobacco and Camelina) to express a suite of biosynthetic enzymes for pheromone precursors production. Several stable transgenic plant lines were produced for the production of high value pheromones, ranging from the carbon chain length of C₁₂ to C₁₆, in either leaves or seeds. These constructed transgenic plant lines produce a significant amount of pheromone precursors in the greenhouse.

In addition to FADs, there are other important enzymes involved in moth pheromone biosynthesis. For example, fatty acyl elongases (ELOs) and acyl-CoA oxidases (ACO) further determine the pheromone skeletons, when combined with FADs. Fatty acyl reductases (FARs) can reduce the pheromone precursors into corresponding alcohol pheromones. To improve the possibility and feasibility of producing customized moth pheromones in plant factories, more functional genes need to be characterized to enlarge the gene toolbox.

The present disclosure discusses the sex pheromone biosynthetic pathways of the beet armyworm Spodoptera exigua and the European grapevine moth Lobesia botrana, which use sex pheromone compounds with two double bonds. Furthermore, in order to clarify the molecular mechanism of moth sex pheromone biosynthesis several genes from three moth species were functionally characterized by encoding corresponding pheromone biosynthetic enzymes, including the genes encoding ELO and ACO. This is the first time for these to be reported in Lepidoptera. These findings improve the feasibility of using plant factories for large-scale customized pheromone production.

Moreover, the final goal of this green chemistry alternative for pest control is to be able to grow the pheromone-releasing plant in the field. Thus, it would be important to obtain plants capable of releasing pheromones directly into the environment. This disclosure discusses an initial effort to explore this possibility with tobacco. A cloned gene promoter called CYP71D16 was used to drive the pheromone biosynthetic genes in tobacco trichomes. A surprising finding was that the pheromone production amount was increased significantly compared to the use of a constitutive promoter.

Compared to other organisms in which the fatty acyl desaturases (FADs) are mostly involved in normal cellular lipid metabolism, moth FADs have evolved extensive functions in the biosynthesis of sex pheromones. Female moths release species-specific sex pheromones to attract conspecific males over a long distance for mating. Moth FADs are key enzymes producing the great diversity of moth sex pheromones. They introduce double bonds in specific positions and with specific geometry in the fatty acyl pheromone precursors.

The present disclosure uses a variety of experimental approaches, including isotope labelling experiments and heterologous expression of gene candidates to characterize several novel FADs involved in pheromone production: The multi-functional SexiDes5 from the beet armyworm Spodoptera exigua and SlitDes5 from the congeneric Spodoptera litura were found to have Δ12 desaturase activities. They use palmitic acid to produce (Z)-11-hexadecenoic acid and the subsequently chain-shortened product (Z)-9-tetradecenoic acid to produce (Z,E)-9,12-tetradecadienoic acid. The European grapevine moth, Lobesia botrana was shown to produce its major pheromone precursor (E,Z)-7,9-dodecanoic acid by an Δ7 FAD. A pheromone gland-specific CsupYPAQ from the rice stem borer Chilo suppressalis was proven to have high activity on palmitic acid to produce (Z)-11-hexadecenoic acid.

The highly evolved moth FADs can be used for production of customized pheromone precursors in transformed organisms for a variety of purposes. Compared to the current conventional synthetic approach which produces hazardous waste during the production process, using semi-synthetic method to produce moth pheromones based on plant-derived pheromone precursors are environmentally friendly. Several plant platforms were utilized to express a suite of biosynthetic enzymes for moth pheromone precursor production. By employing the Agrobacterium-mediated transformation, transgenic Nicotiana spp. and Camelina lines were created for production of C₁₂ to C₁₆ chain length pheromone precursors. The transformed Nicotiana spp. can produce (Z)-11-hexadecenoic acid, (E)-11-tetradecenoic acid, (Z)-11-tetradecenoic acid. The best line from N. benthamiana produced 17.6% (weight %) of (Z)-11-hexadecenoic acid of total fatty acid in vegetative tissue. Also, 7.6% of (E)-9-dodecenoic acid and 6.3% of doubly unsaturated (E,E)-8,10-dodecenoic acid of total fatty acids were produced in seeds of engineered Camelina plants, implying that a significant amount of pheromone precursors might be produced by cultivating these transgenic plants under field conditions.

Knowledge of additional pheromone biosynthetic gene functions can be used to improve the possibility and feasibility of synthesizing customized moth pheromones in plant factories. A fatty acyl elongase (ELO) combined with a Δ11 FAD is considered to provide the fatty acyl pheromone precursors in C. suppressalis. An ELO gene CsupELO4 encoding a protein elongating the major pheromone precursor (Z)-11-hexadecenoic acid into (Z)-13-octadecenoic acid, the precursor of a minor pheromone component, was functionally characterized. This is the first ELO gene that has been functionally characterized in Lepidoptera. The fatty acyl-CoA pheromone precursors are postulated to be reduced and reoxidized to produce the aldehyde pheromone components. Further, CsupFAR2 from C. suppressalis that encodes a fatty acyl reductase (FAR) reducing the major fatty acyl precursors into corresponding fatty alcohols, which are converted into the fatty aldehyde pheromones by followed-up oxidation, was also functionally characterized.

It is further discussed that genetically modified plants that release moth pheromones directly may be used as part of a push-pull strategy. Nicotiana spp. Plants were engineered to release (Z)-11-hexadecenol and (Z)-11-hexadecenyl acetate. The promoter CYP71D16 was cloned, which is a trichome-specific promoter from tobacco Nicotiana tabacum, driving the pheromone biosynthetic genes. A surprising finding was that the production of (Z)-11-hexadecenol increased from 18 to 70 μg per gram fresh leaf when the gene of HarFAR was expressed under CYP71D16 promoter compared to a constitutive promoter CaMV35S.

### Lepidoptera Fatty Acyl Desaturases

The membrane-bound fatty acyl desaturases (FADs) belong to a superfamily of oxygen-dependent membrane di-iron-containing enzymes that includes a conserved three-histidine motif, coordinating two iron ions in the protein active center (Behrouzian and Bruist 2002). The enzymes catalyze the removal of hydrogen from a fatty acyl chain at a specific position resulting in the introduction of double bonds into the chain in ‘E’ or ‘Z’ configuration by desaturation reaction. Unlike the FADs in mammals, plants and protists that are active in normal cellular lipid synthesis, the Lepidoptera FADs have evolved extensively into different functions involved in producing the great diversity of moth pheromones (Knipple et al. 1998; Knipple et al. 2002; Liu et al. 2002; Roelofs et al. 2002; Jeong et al. 2003; Liénard et al. 2010; Tupec et al. 2017).

Since the 1980s, a variety of enzyme activities of moth FADs has successively been reported (Arsequell et al. 1990; Bjostad and Roelofs 1981; 1983; Foster and Roelofs 1988, 1996; Löfstedt and Bengtsson 1988; Martinez et al. 1990; Zhao et al. 1990). Based on the preference of substrates and product differences, particularly the position of the double bond introduced, the moth FADs can be divided into four subfamilies (Tocher et al. 1998).

Over the last three decades, genes encoding corresponding FADs have been characterized via heterologous expression systems, e.g., Δ5 FAD in Ctenopseustis obliquana and C. herana (Hagström et al. 2014), a Δ6 FAD in Antheraea pernyi (Wang et al. 2010), several Δ9 FADs from a range of moth species (Liu et al. 2002; Liu et al. 2004; Rodriguez et al. 2004; Rosenfield et al. 2001), a Δ10 FAD in Planotortrix octo (Hao et al. 2002), a Δ11 FAD in Trichoplusia ni which is the first discovered FAD (Knipple et al. 1998), a Δ11/Δ13 multifunctional FAD in Thaumetopoea pityocampa (Serra et al. 2007), Δ14 FAD in Ostrinia species (Roelofs et al. 2002), and a terminal FAD in Operophtera brumata (Ding et al. 2011), a multifunctional Δ11/Δ12 FADs in two Spodoptera moths, etc. It is known that moth FADs have evolved multiple functions that introduce conjugated double bonds (Moto et al. 2004; Matouškovà et al. 2007; Serra et al., 2006), and produce a triple bond by sequential action (Serra et al. 2007). Still, in many cases, the FADs were reported to use the same substrate but the products have strikingly different stereochemistry (Hao et al. 2002; Liu et al. 2004; Buçek et al. 2015).

The Lepidoptera FADs fall into different groups in the phylogenetic tree. The Δ9 (C₁₆>C₁₈) Glade with a preference for palmitic acid, and the Δ9 (C₁₈>C₁₆) clade with a preference for stearic acid and contain mostly metabolic FADs for maintaining the fluidity of cell membranes. The Δ11/Δ10/Δ9/bifunctional Glade comprises pheromone biosynthetic FADs. The Δ5/Δ6, and Δ14 Glade FADs active in pheromone biosynthesis have a mixture of different signature motifs. In addition, the Δ9 (C₁₄-C₂₆) clade FADs have preferences ranging from myristic acid to long chain (C₁₆) fatty acids and are evolved to produce pheromone compounds as well.

### Moth Pheromones

The name “pheromone” comes from “Pherein” and “Hormon” of Greek origin, means “to carry and to excite” (Wyatt 2003). In 1959, the first moth (Insecta, Lepidoptera) pheromone bombykol (E,Z)-10,12-hexadecadien-1-ol was described from the silk moth Bombyx mori (Butenandt et al. 1959). Pheromones are a subclass of semiochemicals that are used by the individuals of the same species to communicate with each other. Pheromone-mediated behaviors are crucial in animals from insects to mammals and contribute significantly to reproductive isolation (Wyatt 2003; Smadja and Butlin 2009). In many branches in the tree of life, from yeast (Michaelis and Herskowitz 1988) to elephants (Rasmussen et al. 1997) pheromones are used. However, insects are the masters of chemical communication, and most of them heavily depend on pheromones for a wide range of different behaviors (Jurenka 2004; Lamprecht et al. 2008).

Female moths emit species-specific pheromone component blends that attract conspecific males over a long-distance, and this kind of pheromone is called a sex pheromone. Approximately 75% of known moths use Type I sex pheromone compounds, which are C₁₀-C₁₈ fatty acid (FA) derivatives including mainly acetates, alcohols or aldehydes (Löfstedt et al. 2016). The second most common type, Type II pheromones, are used by ca. 15% of the moth species. Type II pheromone compounds comprise polyunsaturated hydrocarbons and their epoxy derivatives with longer straight chains (C₁₇-C₂₅) (Conner et al. 1980; Löfstedt and Kozlov 1997; Ando et al. 2004). The Type I sex pheromone compounds are generally produced in a specialized tissue named the pheromone gland that is commonly located between the 8^{th} and 9^{th} abdominal segments of the female moths (Percy 1987; Raina et al. 2000; Ma et al. 2003; Ando et al. 2004).

Other identified moth sex pheromone compounds are methyl-branched long chain (C₁₇-C₂₃) saturated or unsaturated hydrocarbons, and functionalized hydrocarbons (Type III) (Löfstedt et al. 2016). Also, short-chain secondary alcohols and ketones (Type 0) have been reported not only in Lepidoptera but also in the sister group Trichoptera, and therefore are considered as the most ancient form of moth pheromones (Visser 1986; Löfstedt and Kozlov 1997; Löfstedt et al. 2016), produced in glands of the 5^{th} abdominal sternite (Löfstedt et al. 1994).

### The Biosynthetic Pathways of Moth Pheromones

The general biosynthetic pathways for Type I pheromones from palmitic acid may include chain elongation or shortening, interspersed with desaturation steps to place double bonds in specific positions. Once the chain is completed, the final steps involve adjustment of the terminal functional group (Löfstedt et al. 2016). Biosynthetic pathways for Type II pheromones usually start from linoleic or linolenic acids. In the oenocytes, different chain lengths may be produced and additional double bonds can be introduced similar to the biosynthesis of the Type I pheromone compounds. The final steps involve decarboxylation to provide odd-numbered chains or oxidation followed by decarboxylation and decarbonylation to produce even-numbered chains. The hydrocarbon products are then transported to the pheromone gland for release directly or after epoxidation (Löfstedt et al. 2016). This disclosure is primarily focused on Type I pheromones.

### Identification of Genes Encoding Type I Sex Pheromone Biosynthetic Enzymes

Most Lepidopteran sex pheromones share a common progenitor that is de novo synthesized from acetyl-CoA via fatty acid synthesis in the PG (Foster 2005). The biosynthesis starts by acetyl-CoA carboxylase (ACC) and fatty acid synthase (FAS) catalyzing the saturated fatty acid precursor malonyl-CoA from acetyl-CoA in the first committed biosynthesis step (Volpe and Vagelous 1973; Pape et al. 1988). Fatty-acid metabolism enzymes perform desaturation, chain-shortening by β-oxidation, chain-elongation, and functional group modifications by reduction, acetylation or oxidation to finally produce the pheromone components (Strandh et al. 2008). Different combinations of these enzymes can produce unique species-specific pheromone blends in different species. The genes encoding two classes of essential enzymes involved in moth pheromone synthesis have been mostly functionally identified. Firstly, the gene encoding FADs that introduce double bonds in selected positions of the carbon chains are most extensively studied (Knipple et al. 2002), and has been described in “Lepidoptera fatty acyl desaturases”.

Secondly, the genes encoding fatty-acyl reductases (FAR), responsible for reducing fatty acids to alcohols with different substrate specificities, have been functionally characterized in a few moth species, such as pgFAR-Z/E in O. nubilalis (Lassance et al. 2010), pgFAR in B. mori (Moto et al. 2003) and pgFAR in C. suppressalis.

Other important genes postulated to be involved in moth pheromone production remain to be characterized, including
the genes encoding acetyl-CoA acetyltransferases, which catalyze the conversion of fatty alcohol into acetate ester (Clinkenbeard et al. 1973);
the genes encoding acyl-CoA oxidases, which are responsible for lipid metabolism by catalyzing the conversion of acyl-CoA into trans-2-enoyl-CoA during fatty acid β-oxidation, and two novel ACO genes have been reported in L. botrana;
the genes encoding alcohol oxidases, which are responsible for converting fatty alcohols into aldehydes;
the genes encoding elongation of very long chain fatty acid proteins (ELO), which catalyze the reaction of the long-chain fatty acids elongation cycle, and the first lepidoteran ELO gene has been reported in C. suppressalis;
the genes encoding fatty acid transport proteins, which are integral membrane-bound proteins found in both the plasma membrane and endoplasmic reticulum, several of which facilitate the uptake and activation of exogenous long chain fatty acids (Stahl 2004; DiRusso et al. 2005; Black and DiRusso 2007; Anderson and Stahl 2013); and
the genes encoding acyl-CoA binding proteins, which bind acyl-CoA esters with high specificity and affinity, and are thought to act as intracellular transporters of acyl-CoA esters between different enzymatic systems (Mogensen et al. 1987; Burton et al. 2005; Færgeman et al. 2007).

### Application of Moth Pheromones for Pest Control

Lepidoptera is a large order of insects that contains more than 160,000 described moth and butterfly species, and estimated 250,000 species including undescribed species (Heppner 1991; Nieukerken et al. 2011). Moths are among the most damaging pests of food and fiber crops due to the moths' capability of quickly adapting and evolving resistance to insecticides (Simmons et al. 2010). It should be noted that conventional insecticides will not only hurt the intended pests but are also harmful to other non-target insects, including insects beneficial for pollination or plant protection. Apart from this, in many cases the traditional insecticides are detrimental to humans in regard to food safety and environmental damage (Brittain and Potts 2011).

Due to the variety of problems caused by conventional pesticides, synthetic pheromones emerged as an alternative for insect control by monitoring or disruption of pheromone communication in pest insects with many advantages (Wyatt 2003). Moth pheromones are environmentally friendly and non-toxic, they have no adverse effects on non-target organisms and are not harmful to parasitoids or other beneficial insects. In addition, the risks of resistance being developed in the pest are relatively small. Even in terms of profit and reduction in damage, pheromones often compare favourably to the use of insecticides. For example, protecting cabbage from diamondback moth by pheromone was both cheaper, $62 compared to $123 per ha, and more profitable, ca $800 compared to $456 per ha than by insecticides (Reddy and Guerrero 2000). Nowadays, there are a multitude of synthetic pheromones produced for this application and it is estimated to be about $200 millions pheromone-based control products consumed in the global market (Weatherston and Stewart 2002).

### Plants as Factories for Pheromones Production

Since the techniques for genetically engineering of plants were developed in the early 1980s, numerous research projects have focused on utilizing transgenic plants to produce high-value recombinant proteins or compounds (Boehm 2007; Karg and Kallio 2009; Lienard et al. 2007; Ma et al. 2005; Mett et al. 2008;). During the past 20 years, producing insect pheromones or their biosynthetic precursors in genetically modified plant factories has been attempted. A moth pheromone precursor was produced in Nicotiana tabacum by the introduction of a moth desaturase (Nešněrová et al. 2004), and an aphid alarm pheromone was produced from endogenous plant sesquiterpene by expression of a (E)-β-farnesene synthase in Arabidopsis (Beale et al. 2006). Moreover, Ding et al. (2014) proved that transient expression of genes coding for consecutive pheromone biosynthetic steps in N. benthamiana, resulted in production of biologically active multi-component sex pheromones. The activity of the acetylated sex pheromone mixtures from the fatty alcohol fractions produced by the genetically modified plants have the same activity for trapping of male small ermine moths Yponomeuta evonymella and Y. padella compared to conventionally produced synthetic pheromones (Ding et al. 2014). These studies have demonstrated that it is feasible to produce highly attractive and species-specific moth pheromones in genetically modified plants.

The potential advantages of using plant-based expression systems include the ability to produce complex proteins that require post-translational modifications, avoiding the possibility of introducing human pathogens during the manufacturing process, and the capability to amplify production efficiently and cost-effectively (Ma et al. 2005; Boehm 2007; Liénard et al. 2007; Mett et al. 2008; Karg and Kallio 2009).

### Fatty Acids and Triacylglycerol Biosynthesis in Plants

In plants, de novo fatty acid biosynthesis takes place in plastids (Ohlrogge et al. 1979), which are double-membrane organelles in plant cells. It starts from the condensation of acetyl-coenzyme A (CoA) and malonyl-acyl carrier protein (ACP) by the β-ketoacyl-ACP synthase (KAS) to produce a four-carbon β-ketoacyl-ACP, which are elongated by sequential condensation of two carbon units from malonyl-ACP by the co-operation of enzymes of fatty acid synthase (FAS) (Schultz and Ohlrogge 2001; Voelker and Kinney 2001). Termination of plastid fatty acid chain elongation is catalyzed by fatty acyl-ACP thioesterases (FATs), which hydrolyze acyl chains from ACP to free fatty acids (FFAs). The FFAs are then transported through the plastid and activated to CoA esters, which are assembled into glycerolipids and polar lipids (PL) at the endoplasmic reticulum (ER), where further modifications such as desaturation, hydroxylation, elongation, etc., occur as well.

In developing seeds, the flux of acyl chains in the ER eventually leads to esterification on all three positions of glycerol to form triacylglycerol (TAG). The low polarity of TAG is thought to result in the accumulation of this lipid between bilayer leaflets leading to the budding of storage organelles termed oil bodies (Raclot 1997).

### Fatty Acyl-ACP Thioesterases (FATs)

According to different substrate preferences, FATs are classified into two families, FatA and FatB (Jones et al. 1995; Salas and Ohlrogge 2002). FatAs generally have activities on C₁₈ saturated or unsaturated fatty acyl-ACP, while FatBs are responsible for releasing C₁₆ acyl chain (Sinchez et al. 2010). FatA orthologues show high activity upon Z9-18-ACP substrate, of which the substrate specificities are similar among different species (Hawkins and Kridl 1998; Knutzon et al. 1992). While FatB enzymes can be further classified into two subclasses, the first is FatB1 that has generally preference for 16:0-ACP, and the second is FatB2 that prefer short- and medium-chain saturated acyl-ACPs (Rodriguez et al. 2014).

FATs are the key enzymes to determine which fatty acids are exported to the cytosol and subsequently incorporated into further glycerolipids biosynthesis (Voelder T A et al. 1996). To date, a variety of specific FAT genes have been functionally demonstrated to effectively modify oil profile in transgenic plants (Salas and Ohlrogge 2002). The engineered fatty acids by FAT genes range from short-chain to long-chain. For instance, e.g., overexpression of FatB2 originally from California bay laurel (Umbellularia californica) in Brassica napus and Camelina seeds increased the lauric acid level in the total fatty acids (mol %) from negligible level to 58% (Voelker et al. 1996) and 29% (Kim et al. 2015), respectively; FatB2 from Cuphea palustris overexpressed in N. benthamiana leaf boosts the production of myristic acid a hundred times (Ding et al. 2014); B. napus introduced by FatB1 from Cuphea species produced 34% palmitic acid of total fatty acids (mol %) (Jones et al. 1995). Interest in the use of FATs in lipid biotechnology has led to a very active research on their different forms coming from various sources (Mandal et al. 2000; Othman et al. 2000; Serrano et al. 2005; Ghosh et al. 2007).

### Metabolic Pathway of Medium-Chain Fatty Acid Synthesis in Plant

Medium-chain fatty acids (MCFAs) range from ethanthic acid (C6:0) to myristic acid (C14:0), which are important for a variety of industrial productions, such as cosmetics, detergents, soaps, surfactants, lubricants, etc (Knaut and Richtler 1985; Dyer et al. 2008). The synthesis of MCFAs is a variation on typical de novo fatty acid synthesis that takes place in plants that produces primarily C₁₆ and C₁₈ fatty acids. In nature, only a few plants are MCFA-rich. Therefore, engineered pathways are usually applied to generate MCFAs in non-MCFA-enriched plants.

The MCFA enriched plants are mostly from the tropics, e.g., palm kernel (Elaeis guineensis Jacq.) contains ca. 50 (mol) % of lauric acid and 18% of myristic acid of total fatty acids, respectively, as well as coconut (Cocos nucifers L.). The seeds from the temperate Cuphea genus also produce high amounts of MCFAs (Graham and Kleiman 1992; Graham 1998), of which C. pulcherrima can yield more than 90% of C8:0, and C. viscosissima accumulates 25% of C8:0 and 64% of capric acid (C10:0). Therefore, Cuphea species have been a suitable genetic resource to isolate FAT genes for MCFA production. Establishing oilseed crop lines for MCFA production by introducing Cuphea FAT genes have been confirmed to be a useful approach (Dehesh et al. 1996a, b; Leonard et al. 1997; Slabaugh et al. 1998; Filichkin et al. 2006).

For purposes of increasing MCFA content, divergent FatB enzymes were characterized and transgenically investigated, predominantly focusing on the engineering of lauric acid (C12:0) (Eccleston and Ohlrogge 1998; Knutzon et al. 1999; Voelker et al. 1992; Reynolds et al. 2017). In plants, the biosynthesis of MCFA is a variation on typical de novo fatty acid synthesis that generates primarily C₁₆ and C₁₈ fatty acids. Chain-lengths of fatty acids are primarily determined by acyl-ACP thioesterases, in including FatB thioesterases that typically release C₁₆ acyl chains from de novo fatty acid biosynthesis (Li-Beisson et al. 2013). Variant forms of FatB, found in selected plant species, are able to release fatty acids of chain lengths shorter than C₁₆, as demonstrated by transgenic expression in seeds (Pollard et al. 1991; Jones et al. 1995; Voelker 1996; Tjellström et al. 2013; Kim et al. 2015). In a previous study, a FatB gene UcTE from California bay laurel (Umbellularia californica) was found to have high activity for production of 12:0 in rapeseed (Brassica napus) (Voelker et al. 1992). When the MCFAs are exported into the cytoplasm from the plastid in oilseeds, they become available for incorporation into TAG, which is formed most directly by the Kennedy pathway enzymes of glycerol-3-phosphate acyltransferase (GPAT), lysophosphatidic acid acyltransferase (LPAAT) and diacylglycerol acyltransferase (DGAT) continuously (Thelen and Ohlrogge 2002; Cahoon et al. 2007; Dyer et al. 2008; Kim et al. 2015a). A recent report showed that the co-expression of a variant FatB thioesterase with LPAT in Camelina seeds, the MCFA accumulation was improved (Kim et al. 2015a).

As such, there is a need for improved methods with increased production of insect pheromones and insect pheromone precursors in plant factories.

### BRIEF SUMMARY

In accordance with one embodiment of the present disclosure, there is contemplated a variety of experimental approaches, including isotope labelling experiments and heterologous expression of gene candidates to characterize several novel FADs involved in pheromone production: The multi-functional SexiDes5 from the beet armyworm Spodoptera exigua and SlitDes5 from the congeneric Spodoptera litura were found to have Δ12 desaturase activities. They use palmitic acid to produce (Z)-11-hexadecenoic acid and the subsequently chain-shortened product (Z)-9-tetradecenoic acid to produce (Z,E)-9,12-tetradecadienoic acid. The European grapevine moth, Lobesia botrana was shown to produce its major pheromone precursor (E,Z)-7,9-dodecanoic acid by an Δ7 FAD. A pheromone gland-specific CsupYPAQ from the rice stem borer Chilo suppressalis was proven to have high activity on palmitic acid to produce (Z)-11-hexadecenoic acid.

The highly evolved moth FADs can be used for production of customized pheromone precursors in transformed organisms for a variety of purposes. Compared to the current conventional synthetic approach which produces hazardous waste during the production process, using semi-synthetic method to produce moth pheromones based on plant-derived pheromone precursors are environmentally friendly. The present disclosure investigated the use of several plant platforms to express a suite of biosynthetic enzymes for moth pheromone precursor production. By employing the Agrobacterium-mediated transformation, transgenic Nicotiana spp. and Camelina lines were constructed for production of C₁₂ to C₁₆ chain length pheromone precursors. The transformed Nicotiana spp. can produce (Z)-11-hexadecenoic acid, (E)-11-tetradecenoic acid, (Z)-11-tetradecenoic acid. The best line from N. benthamiana produced 17.6% (weight %) of (Z)-11-hexadecenoic acid of total fatty acid in vegetative tissue. Also, 7.6% of (E)-9-dodecenoic acid and 6.3% of doubly unsaturated (E,E)-8,10-dodecenoic acid of total fatty acids were produced in seeds of engineered Camelina plants, implying that a significant amount of pheromone precursors might be produced by cultivating these transgenic plants under field conditions.

Knowledge of additional pheromone biosynthetic gene functions can be used to improve the possibility and feasibility of synthesizing customized moth pheromones in plant factories. A fatty acyl elongase (ELO) combined with a Δ11 FAD is considered to provide the fatty acyl pheromone precursors in C. suppressalis. An ELO gene CsupELO4 was functionally characterized encoding a protein elongating the major pheromone precursor (Z)-11-hexadecenoic acid into (Z)-13-octadecenoic acid, the precursor of a minor pheromone component. This is the first ELO gene that has been functionally characterized in Lepidoptera. The fatty acyl-CoA pheromone precursors are postulated to be reduced and reoxidized to produce the aldehyde pheromone components. Also characterized was CsupFAR2 from C. suppressalis that encodes a fatty acyl reductase (FAR) reducing the major fatty acyl precursors into corresponding fatty alcohols, which are converted into the fatty aldehyde pheromones by followed-up oxidation.

Genetically modified plants actually releasing moth pheromones may be used as part of a push-pull strategy. Nicotiana spp. Plants were engineered to release (Z)-11-hexadecenol and (Z)-11-hexadecenyl acetate. The promoter CYP71D16 was cloned, which is a trichome-specific promoter from tobacco Nicotiana tabacum, driving the pheromone biosynthetic genes. The surprising finding was that the production of (Z)-11-hexadecenol increased from 18 to 70 μg per gram fresh leaf when the gene of HarFAR was expressed under CYP71D16 promoter compared to a constitutive promoter CaMV35S.

To understand the molecular mechanisms of moth pheromone biosynthesis and extension of the synthetic gene pool will be helpful in order to design tailor-made production of moth pheromones in transgenic organic factories. One significant aim of this disclosure is to characterize important enzymes involved in pheromone biosynthesis, especially new FADs that are specialized in performing distinct functions and providing significant molecular evidence for the study about the sequences to functions of FADs.

The second major aim of this disclosure is finding synthetic biology methods to further demonstrate the technical and commercial feasibility of insect pheromone production in plant factories by stable transformation. The proposed strategy has the potential to become an economically sound part of many integrated pest management (IPM) programs. The concept of using transient expression of the necessary and sufficient genes for production of common moth pheromone compounds in Nicotiana benthamiana has been proven in a previous study (Ding et al. 2014). This synthetic biology strategy is a “green chemistry”-alternative, which aims for a novel and cost-effective way of producing moderate to large quantities of pheromones with high purity and a minimum of waste. Synthetic biology can be explained as the studies to take the rational design principles of engineering and apply them to the modification and manipulation of living organisms (Gibbs 2004). This has resulted in the construction of increasingly complex genetic circuits and rewired pathways, although the manual construction of these circuits can often be a time-intensive task with complex optimization required (Cloney 2016).

The disclosure is divided into two major parts, the characterization of novel genes involved in pheromone biosynthesis and the construction of gene cassettes for insect pheromone production in plants. The objective of the first part is deciphering the molecular mechanism of pheromone biosynthesis in three moth species that are notorious pests, i.e., Spodoptera exigua, Lobesia botrana, and Chilo suppressalis. The second part is dedicated to designing, building and assembling an integrated biological system to manufacture insect pheromones in plant factories. In this part, the goal was to produce stable lines of transformed plants for the production of C₁₄ and C₁₆ and C₁₂ insect pheromone precursors. For the purpose of optimizing the plant factories, storage and release of pheromone compounds from the modified plants was also investigated.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 depicts lab results of a genetically modified plant as described herein; and

FIG. 2 is a diagram of certain gene cassettes used to modify plants as described herein.

### DETAILED DESCRIPTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

### Plant Platforms for Pheromone Production

In this disclosure, three different plant platforms were utilized: N. tabacum, N. benthamiana and Camelina sativa for pheromone production.

N. tabacum is also called cultivated tobacco which is an herbaceous plant and it is only found in cultivation. N. tabacum is the most commonly grown plant in the Nicotiana genus. It is commercially grown in many countries and the leaves are used to produce tobacco. The height of matured tobacco plants is between 1 and 2 meters. The leaves vary in size and the lower leaves are the largest with a length of up to 60 cm. N. benthamiana is a close relative of N. tabacum, and the mature plants show a large variation in height, ranging from as tall as 1.5 meters to shorter than 200 mm. In our greenhouse, the height of N. benthamiana was about 300 mm. The two Nicotiana species are both favourable to work with in metabolic engineering aiming at production of pheromone compounds as they have relatively short production times, large area of leaves to output volatiles and are relatively easy to grow in controlled growth conditions. In addition, there is less concern about contaminating food supplies as they are not food crops.

Camelina was chosen as the oilseed production platform for our studies because it is limited use as a food crop and is considered an ideal system for rapid introduction and evaluation of fatty acid and other oil-related traits (Iskandarov et al. 2014). Foremost, transgenes can easily be introduced into Camelina using a simple Agrobacterium-based method (Lu and Kang 2008), and it has a relatively short life cycle that allows up to three generations in a year for evaluation of engineered traits (Bansal and Durrett 2016). Camelina is also closely related to Arabidopsis thaliana, with a wealth of transgenic and genomic data for optimizing endogenous biosynthetic pathways for production of desired oil traits in seeds that typically are 30% to 40% oil by weight (Nguyen et al. 2013).

In many plants, trichomes are tiny specialized hair structures for secondary metabolite production and release. For instance, biosynthesis of the diterpenes takes place in trichome heads, where secretory vesicles and cells are located (Kandra and Wagner 1988; Duke 1994; Guo et al. 1995). CYP71D16 was confirmed as a trichome-specific promoter leading the downstream gene to be specifically expressed in plant trichome (Wang et al. 2002). To explore the possibility of releasing moth pheromones from Nicotiana leave's trichome cells, a N. tabacum trichome specific promoter pCYP71D16 was used for driving pheromone biosynthetic gene expression.

### General Methodology

### Labelling Experiment

Isotopic labelling was the technique used to identify moth pheromone biosynthetic pathways by tracking the passage of an isotope through the metabolic pathways in this disclosure. The deuterium-labelled precursors were separately dissolved in dimethylsulphoxide (DMSO) (Bjostad and Roelofs 1983; Yamaoka et al. 1984) and topically applied to the female abdominal tip where the pheromone gland is located. After incubation of a half to several hours, pheromone glands were excised and extracted (Bjostad and Roelofs 1984), and the samples could be analyzed by gas chromatography/mass spectrometry (GC/MS) (Christie 1998). The double bond positions in the fatty acid chain were confirmed by dimethyl disulfide (DMDS) derivatization (Dunkelblum et al. 1985).

### Cloning and Plasmid Constructs for Assays

Amplification of genes was performed by PCR either from cDNA templates synthesized from total RNA or genome DNA extracted directly from plant materials (for trichome specific promoter pCYP71D16). Custom DNA synthesized by Invitrogen was used for some genes. All the genes contain the Gateway® cloning site attB (Gateway cloning system, Invitrogen) and were subsequently cloned to Gateway® entry vector in first step by BP reaction. For constructing co-expression clones, the Phusion PCR (Atanassov et al. 2009) was performed by putting two fragments that containing several dozens of homologue sequence bases and the DNA polymerase together. After hybridizing of the two fragments, the recombined sequence was cloned to Gateway® vector. The expression clones in this disclosure were also constructed by using Gateway® method (Katzen 2007). After the entry clones were confirmed by sequencing (Schuster 2007), all of them were sub-cloned to a destination vector by LR reaction with different multigene combinations for a variety of purposes.

### Functional Assays

In this disclosure, we have used three platforms for gene functional characterization. One was the yeast expression system. The yeast expression vectors pYEX-CHT and pYES52 were used for the functional assays. The expression clones containing FADs were introduced into the double deficient ole1/elo1 strain (MATa elo1::HIS3 ole1::LEU2 ade2 his3 leu2 ura3) of the yeast Saccharomyces cerevisiae, while the expression clones containing FARs were introduced into the INVSc strain of yeast S.c. (MATa HIS3 LEU2 trp1-289 ura3-52). The transformation of yeasts was carried out using the S.c. easy yeast transformation kit (Life technologies).

The second platform for gene functional assay was the plant transient expression system. In this, we used N. benthamiana as the plant platform for gene expression. The plant expression clones in pXZY393 vector containing the target genes were first introduced into Agrobacterium tumefaciens GV3101 strain (MP90RK) by electroporation (1700 V mm⁻¹, 5 ms, Eppendorf 2510). Meanwhile, a viral silencing suppressor protein P19 was introduced into the same A. tumefaciens strain as well in order to inhibit the transgene silencing of the host cells and extend transgene expression over a longer period of time with a higher degree of expression (Voinnet et al. 2003). Subsequently the transformed A. tumefaciens was incubated for several days until the culture concentration was high enough for infiltration of N. benthamiana. The infiltration experiment was carried out by using a 1 mL syringe without needle, containing the A. tumefaciens cells, to inject the lower side of a suitable four-week-old N. benthamiana leaf, with a gentle squeeze on the plunger and modest pressure on the leaf using a finger.

The last platform we used for gene functional assay was the insect cell expression system. The expression construct for candidate gene in the BEVS donor vector pDEST8 was made by LR reaction. Recombinant bacmids were made according to instructions for the Bac-to-Bac™ system given by the manufacturer Invitrogen using DH10MEmBacY (Geneva Biotech). Baculovirus generation was done using Sf9 cells (Invitrogen), Ex-Cell 420 medium (Sigma) and baculoFECTIN II (OET).

### Leaf Disc Transformation Via Agrobacterium

The method of Agrobacterium-mediated leaf disc transformation (Clemente et al. 2006) was used for Nicotiana spp. stable transformation. First, the A. tumefaciens culture containing the construct was incubated at 30° C. in LB medium supplemented with suitable antibiotics, until the optical density (A600 nm) can be adjusted to 0.9-1. Plant material was obtained from 4-5 weeks old Nicotiana plants grown under sterile conditions on MS medium (Murashige and Skoog 1962) in a climate chamber. Subsequently, the transgenic lines were obtained by Agrobacterium-mediated leaf-disc transformation. Leaf discs (20 mm×20 mm) were cut out and incubated 5 min in an A. tumefaciens solution, dried with sterile napkin paper and transferred to Petri dishes with MS medium (Horsch el al. 1985). After 2-3 days incubation in darkness, leaf discs were transferred to selection medium. Then after 2-3 weeks of incubation, the callus produced on the leaf edges were transferred to shoot-inducing medium. After 2-3 weeks of incubation, the shoots were transferred to root-inducing medium. The shoots were finally transferred into soil and grown in greenhouse until maturity.

### Floral Dip Transformation Via Agrobacterium

The method of Agrobacterium-mediated floral dip transformation was used for Camelina stable transformation. The constructed expression vectors were introduced into Agrobacterium tumefaciens strain GV3101 (MP90RK) by electroporation (1700Vmm⁻¹, 5 ms, Eppendorf 2510). The transformed Agrobacterium cells were grown on solid LB medium supplemented with antibiotics (50 mg/L rifampicin, 50 mg/L gentamicin and 50 mg/L spectinomycin) after incubating at 30° C. for 36 h. Afterwards, a single clone from each expression clone was incubated in 2 mL liquid LB medium with suitable antibiotics as described above at 30° C. for 36 h. Then the Agrobacterium solution was transferred to 30 mL medium for a 36 h incubation, and after that, the solution was transferred to 1 L medium for a 24 h incubation. Subsequently, the five weeks old Camelina plants were then transformed (Lu and Kang 2008) by the floral dip/vacuum infiltration method as described in Liu et al. (2012). After eight to ten weeks, the seeds were collected and sown in soil, and herbicide was used to select the transformants.

### Lipid Analysis

In order to analyze the gene functions in transformed yeast, insect cells and plants, as well as to detect the pheromone precursor production in Nicotiana spp. leaves and Camelina seeds, the lipid analysis was performed. For the lipid analysis, first the pheromone gland tissues, yeast cells, insect cells or plant leaves were extracted by chloroform:methanol (2:1 v:v) at room temperature overnight. For fatty acids (pheromone precursors) analysis, this was followed by base-methanolysis and transesterification as previously described (Dunkelblum and Kehat 1987) to convert all fatty acids to their corresponding methyl esters. The products were then recovered in n-heptane prior to GC/MS analysis. While the samples for pheromone compounds such as alcohol, aldehyde and acetate analysis were followed by adding n-heptane to re-dissolve the compounds after the total lipid dryness by nitrogen, which were then transferred to new glass vials for GC/MS analysis. The seed fatty acids extraction and methanolysis was performed directly by adding 2% sulphuric acid in methanol and incubated at 90° C. for 1 h, and then water and n-heptane were added to extract the fatty acid methyl ester products prior to GC/MS analysis.

### Headspace Volatile Collection

To perform the experiment of plant headspace volatile collection, the experiment was carried out as described in Raguso and Pellmyr (1998). For the use of solid phase micro extraction (SPME) to collect volatile, the experiment was performed as described in Centini et al. (1996).

### Results and Discussion

### Labelling Experiments Reveal New FADs Activities

Tracking the pheromone biosynthetic pathways of S. exigua and L. botrana by isotope-labelling experiment, we confirmed an unusual Δ12 and a novel Δ7 desaturation activity to be involved, respectively. In the S. exigua, the in vivo labelling experiment showed that deuterium atoms from [16,16,16-²H_{3]} hexadecanoic acid (D₃-16:acid) were incorporated into all the detected acetates and alcohols, including (Z)-11-hexadecenyl acetate (Z11-16:OAc), (Z)-11-hexadecenol (Z11-16:OH), (Z)-9-tetradecenyl acetate (Z9-14:OAc), (Z)-9-tetradecenol (Z9-14:OH), (Z,E)-9,12-tetradecadienoic acetate (Z9,E12-14:OAc) and (Z,E)-9,12-tetradecadienol (Z9,E12-14:OH), as well as into the corresponding FAMEs, i.e., methyl (Z)-11-hexadecanoate (Z11-16:Me), methyl (Z)-9-tetradecenoate (Z9-14:Me), methyl (Z,E)-9,12-tetradecadienoate (Z9,E12-14:Me) and methyl myristate (14:Me). Notably, when non-labeled (Z)-9-tetradecenoic acid (Z9-14:acid) was applied to the pheromone gland, the amount of Z9,E12-14:OAc increased significantly. In contrast, when [14,14,14-²H₃] tetradecanoic acid (D₃-14:acid) and (E)-12-[14,14,14-²H₃] tetradecenoic acid (D₃-E12-14:acid) were applied, label incorporation was not detected in any of the above components. These results confirm that the production of major pheromone component Z9,E12-14:OAc in S. exigua starts from palmitic acid and a Δ11 FADs acts on palmitic acid to produce Z11-16:acid, which is then chain-shortened to Z9-14:acid, followed by the second desaturation at the Δ12 position to form Z9,E12-14:OAc (This pathway is in line with previous study of S. littoralis (Jurenka, 1997).

Deuterium labelled D₃-16:acid, D₃-14:acid, (Z)-11-[13,13,14,14,14,-²H₃] tetradecenoic acid (D₅-Z11-14:acid), (Z)-9-[12,12,12-²H₃] dodecenoic acid (D₃-Z9-12:acid) and [12,12,12-²H₃] dodecanoic acid (D₃-12:acid) were topically applied to the pheromone glands of L. botrana to track the biosynthetic pathway. The results showed that deuterium atoms from D₃-16:acid were incorporated into all the pheromone components, including (Z)-9-dodecenyl acetate (Z9-12:OAc), (E,Z)-7,9-dodecadienol (E7,Z9-12:OH) and (E,Z)-7,9-dodecadienyl acetate (E7,Z9-12:OAc). In the fatty acyl compounds analysis, deuterium atoms from D₃-16:acid were incorporated into methyl palmitate (16:Me), 14:Me, methyl (Z)-11-tetradecanoate (Z11-14:Me), methyl lauritate (12:Me) and methyl (E,Z)-7,9-dodecadienoate (E7,Z9-12:Me). Application of D₃-14:acid showed the same incorporation results as observed for D₃-16:acid, whereas no label incorporation was found from D₃-14:acid into 16:acid. Labels from D₅-Z11-14:acid were incorporated into Z9-12:OAc, E7,Z9-12:OH and E7,Z9-12:OAc, as well as into Z9-12:Me and E7,Z9-12:Me intermediates. Additionally, labels from D₃-Z9-12:acid were extremely highly incorporated into E7,Z9-12:OAc and incorporated into Z9-12:OAc and E7,Z9-12:OH as well. In contrast, when D₃-12:acid was applied, label incorporation was not detected in any of the above mentioned compounds. The incorporation of deuterium labels from D₃-Z9-12:acid into E7,Z9-12:OAc, E7,Z9-12:OH, and E7,Z9-12:Me strongly suggests an elusive Δ7 desaturation on Z9-12:acid to form the major pheromone precursor E7,Z9-12:acid in L. botrana.

### Functional Assays of Novel FAD Genes

Here is disclosed several novel FAD genes with a variety of functions. First, involving the Δ12 desaturation activity, two unusual Δ11/Δ12 FAD genes SexiDes5 and SlitDes5 were characterized in S. exigua and the congeneric S. litura, with the same functions using palmitic acid and the subsequently chain-shortened product (Z)-9-tetradecenoic acid (Z9-14:acid) as substrates to produce (Z,E)-9,12-tetradecadienoic acid (Z9,E12-14:acid), respectively. A Δ11 FAD gene Lbo_PPTQ from L. botrana can produce important pheromone intermediate Z11-14:acid from myristic acid. C. suppressalis utilizes (Z)-11-hexadecenal (Z11-16:Ald) as its major pheromone component. By heterologous expression in yeast and plants, a Δ11 FAD gene CsupYPAQ presents very high substrate specificity to palmitic acid for production of (Z)-11-hexadecenoic acid (Z11-16:acid) with great activity. Another FAD gene CsupKPSE from C. suppressalis has preference for C₁₆. It is interesting, however, that CsupKPSE switches the preference for C₁₆ to C₁₈ to form oleic acid when the culture nutrition was limited.

The predicted ER retention signature motif in Δ11/Δ12 FADs SexiDes5, SlitDes5, and Δ11 FAD CsupYPAQ is “LPAQ”, “LPSQ”, and “YPAQ” respectively. It is noticed that the motif difference in FADs is to some extent related to the functions, e.g., FADs within the “KPSE” group are Δ9 desaturases having preference for C₁₆, while the “NPVE” group are mainly modifying C₁₈ (Rosenfield et al. 2001; Liu et al. 2002; Liu et al. 2004; Rodriguez et al. 2004). The “xxxQ” are most likely to be the Δ11, Δ10 and multi-functional FADs and a few exceptions are Δ9 FADs, which are exclusively involved in pheromone biosynthesis (Knipple et al. 1998; Hao et al. 2002; Serra et al. 2007; Xia et al. 2015). The Δ6 and Δ14 groups contain a mixture of different signature motifs from the Δ9 and Δ11 groups, and their biological functions are diverged from Δ9, and Δ11 FADs (Roelofs et al. 2002; Wang et al. 2010). On the other hand, there were some exceptions to the “motif” signal hypothesis. For example, Ding et al. (2016) reported that one amino acid at the cytosolic carboxyl terminus of the protein (258E), which is outside of the motif region, is critical for the ‘Z’ activity of the Choristoneura rosaceana FAD.

In this disclosure, SexiDes5 and SlitDes5 were showing same functions in the yeast although with different ER motifs. The motif of CsupYPAQ has only one amino acid different from SexiDes5. However, CsupYPAQ is highly specific to palmitic acid, whereas SexiDes5 can form a variety of products with wide preferences for both saturated and unsaturated fatty acids. Thus, making mutations at different sites for these unique FADs could be a possible approach to explore the relationship between sequences and functions of FADs.

### Production of Pheromone Precursors in Plants

Many functionally diversified FADs were characterized by previous studies, making it possible to design tailor-made pheromone precursor production in engineered plants. In this disclosure, N. tabacum, N. benthamiana and C. sativa were successfully transformed to obtain stable transgenic lines producing pheromone precursors.

The successful production of several insect sex pheromone precursors in N. tabacum and N. benthamiana by stable transformation was demonstrated. Levels of up to 17.6% Z11-16:acid in the total fatty acids was achieved. All of the transformed plastid FatBs and FADs were functionally active in producing pheromone precursor in both Nicotiana species. This is the first report of Z/E11-14:acid production in a plant by stable transformation. The performance of Z11-16:acid production in N. benthamiana was better than in N. tabacum. The average value of Z11-16:acid production in T0 N. tabacum was 0.2% whereas in T0 N. benthamiana it was 1.8%. The best N. benthamiana transgenic line #025 produced as high as 13.6% of Z11-16:acid of the total fatty acids in T2 plants, which is much higher than the production amount reported from Nešnerová a et al. 2004, claiming that 6% of Z11-16:acid of total fatty acids was produced in their transgenic N. tabacum NtD15B line. The quantity of Z11-16:acid in the form of methyl ester in N. benthamiana determined to be 335 μg per gram fresh leaf is also higher, compared to 32 μg per gram of N. tabacum fresh leaf in Nešnerová et al. 2004. The results suggest that N. benthamiana has potential to be more efficient than N. tabacum as a plant factory for Z11-16:acid production. In the report of Ding et al. 2014, 381 μg per gram fresh leaf of Z11-16:acid was produced in N. benthamiana by transient expression, which is a massive overexpression of exogenous genes over a few days that ignores the health of the plant. Here, the production of 335 μg per gram leaf of Z11-16:acid by stable transformation shows the ability of vegetative material to function with the expression of CpuFatB1 and AtrΔ11 and yield compounds over development, providing the potential for further commercial production.

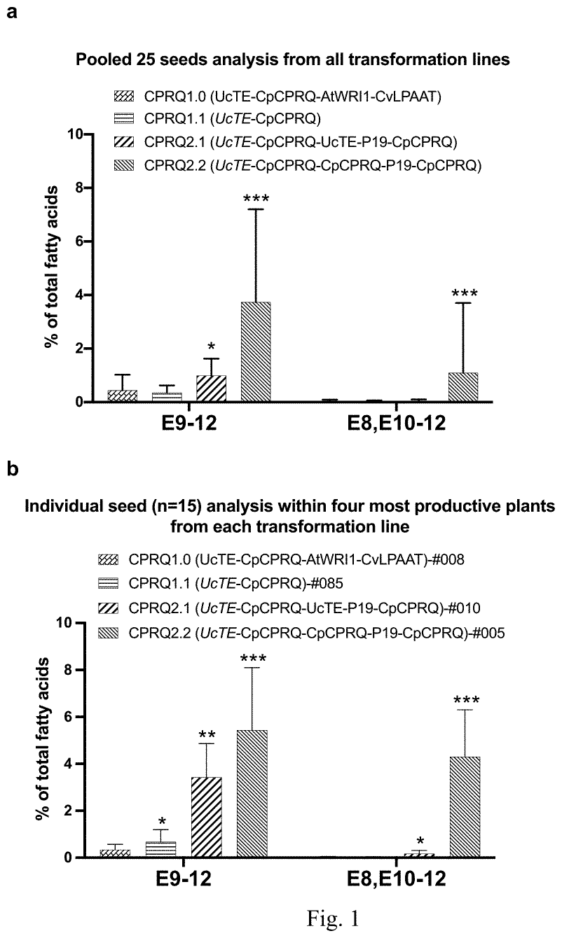

To investigate the feasibility of a “plant factory production” in Camelina, we established four different types of transformant lines by using different exogenous gene cassettes, as described below. In the regenerated Camelina seeds, the mono-unsaturated E9-12:acid with small amount of (Z)-9-dodecenoic acid (Z9-12:acid) and diunsaturated E8,E10-12:acid were produced in all the four types of transformant lines, as shown in FIG. 1. Camelina was genetically modified for production of E8,E10-12:acid via stable integration of gene cassettes using Agrobacterium-mediated floral dip transformation. This is the first report on production of di-unsaturated pheromone precursors in plants. Furthermore, the production amount of 6.3% of E8,E10-12:acid of total fatty acids is quite high. Because the oil content of the Camelina seeds, on a dry weight basis, is typically between 35 to 45% and the yields of Camelina are in a range of 336 to 2240 kg of seeds per hectare (Moser 2010). This means that 7.4 to 63.5 kg (minimum to maximum) of E8,E10-12:acid might be produced by cultivating our best Camelina line. Moreover, in this study we also investigated four strategies for optimization of the plant factory for production of E8,E10-12:acid. We demonstrated that co-expression of the desaturase with P19 and multiple gene copies can increase the production of C₁₂ pheromone precursors significantly. Also, it was confirmed that stably expressing P19 regulated by the seed-specific napin promoter would not cause observable harm of plant development.

Our studies described here explore the use of an oilseed crop Camelina sativa (Camelina) as a production platform for C₁₂ pheromone precursors. To optimize the seeds of oil crops for the production of these precursors requires redesigning fatty acid biosynthesis to generate maximal levels of 12:0, rather than the typical C₁₆ and C₁₈ fatty acids found in triacylglycerols (TAG) of these seeds. In plants, the biosynthesis of MCFA is a variation on typical de novo fatty acid synthesis that generates primarily C₁₆ and C₁₈ fatty acids. Chain-lengths of fatty acids are primarily determined by acyl-ACP thioesterases, including FatB thioesterases that typically release C₁₆ acyl chains from de novo fatty acid biosynthesis (Li-Beisson et al. 2013). Variant forms of FatB, found in selected plant species, are able to release fatty acids of chain lengths shorter than C₁₆, as demonstrated by transgenic expression in seeds (Pollard et al. 1991; Jones et al. 1995; Voelker 1996; Tjellström et al. 2013; Kim et al. 2015). In previous study, a FatB gene UcTE from California bay laurel (Umbellularia californica) was found to have high activity for production of 12:0 in rapeseed (Brassica napus) (Voelker et al. 1992). When the MCFAs are exported into the cytoplasm from the plastid in oilseeds, they become available for incorporation into TAG, which is formed most directly by the Kennedy pathway enzymes of glycerol-3-phosphate acyltransferase (GPAT), lysophosphatidic acid acyltransferase (LPAAT) and diacylglycerol acyltransferase (DGAT) continuously (Thelen and Ohlrogge, 2002; Cahoon et al. 2007; Dyer et al. 2008; Kim et al. 2015a). A recent report showed that the co-expression of a variant FatB thioesterase with LPAT in Camelina seeds, the MCFA accumulation was improved (Kim et al. 2015a). In addition, over-expression of the seed specific transcription factor WRINKLED1 from Arabidopsis was confirmed a useful approach to elevate the TAG concentration (Cernac and Benning 2004).

Raising the target compound in transgenic engineering plants by transformation of multiple transgene copies also have been reported in some studies (Carrier et al. 1998; Mansur et al., 2005; Nguyen et al., 2015; Schultz et al. 1987). This approach may be hampered by the observation that the silencing effect of the transgenes could be increased (Matzke et al. 1994; Tang et al. 2006). However, new biotechnological ways have been reported to avoid the gene silencing effect by using different promoter for each gene copy as well as codon optimization (Fath et al. 2011). Moreover, Naim et al. (2016) informed that stable expression of silencing-suppressor proteins (SSPs) such as P19, V2, P0 could enhance the performance and longevity of a metabolic pathway.

In this study, we engineered the pathways towards to the production of the major fatty acyl pheromone precursor, E8,E10-12:acid, of the codling moth Cydia pomonella in Camelina seeds. Camelina was chosen as the oilseed production platform for our studies because it is limited use as a food crop and is considered an ideal system for rapid introduction and evaluation of fatty acid and other oil-related traits (Iskandarov et al. 2014). Foremost, transgenes can easily be introduced into Camelina using a simple Agrobacterium-based method (Lu and Kang 2008), and it has a relatively short life cycle that allows up to three generations in a year for evaluation of engineered traits (Bansal and Durrett 2016). Camelina is also closely related to Arabidopsis thaliana, with a wealth of transgenic and genomic data for optimizing endogenous biosynthetic pathways for production of desired oil traits in seeds that typically are 30% to 40% oil by weight (Nguyen et al. 2013).

To explore oilseed production of high amount of C₁₂ pheromone precursors, we co-expressed the UcTE and CpCPRQ in Camelina seeds for production of (E)-9-dodecenoic acid (E9-12:acid) and (E,E)-8,10-dodecadienoic acid (E8,E10-12:acid). We investigated four strategies towards the production of these two compound in the plant factory: 1) co-expression of a seed-specific synthetic transcription factor WRINKLED1 from Arabidopsis thaliana enhancing fatty acid synthesis and a Kennedy pathway gene, lysophosphatidic acid acyltransferase gene CvLPAAT from Cuphea viscosissima; 2) transformation of only one gene copy of CpCPRQ into high lauric Camelina seeds; 3) transformation of multiple gene copies with different promoters and terminators; and 4) stable expression of the viral gene silencing suppressor protein P19 together with pheromone biosynthetic genes. Our findings demonstrated the feasibility of producing the codlemone precursor and other C₁₂ pheromone precursors in stably transformed plant seeds.

### Plant Material and Growth Conditions

For these studies, Camelina sativa cv. Suneson (Camelina) was used. The previously described high lauric (20 mol % of total seed fatty acids) Camelina line was used as our primary metabolic engineering platform (Kim et al. 2015a). Two plants per pot with soil (Krukväxtjord lera & kisel) were grown under greenhouse conditions of 24° C., 14 h day/18° C., 10 h night, with supplemental lighting.

### Gene Cloning—Preparation of Constructs

UcTE (Genebank access number: Q41635.1), codon optimized-CpCPRQ opening reading frames (ORFs), seed-specific promoters for the α′-subunit of β-conglycinin gene (β-con) (Chamberland et al. 1992), β-phaseolin (β-Phaseolin) (Geest and Hall 1996), oleosin gene (Oleosin) (Fan et al. 2013), nopaline synthase terminator (NOS), and nopaline synthase terminator (HSP) were synthesized by Invitrogen. ORF of CpCPRQ (AHW98354), AtWRINKLED1 (AY254038), CvLPAAT (ALM22867), P19 (P69516.1), and sequence of napin gene promoter (napin), octopine synthase terminator (OCS) were amplified from entry clones.

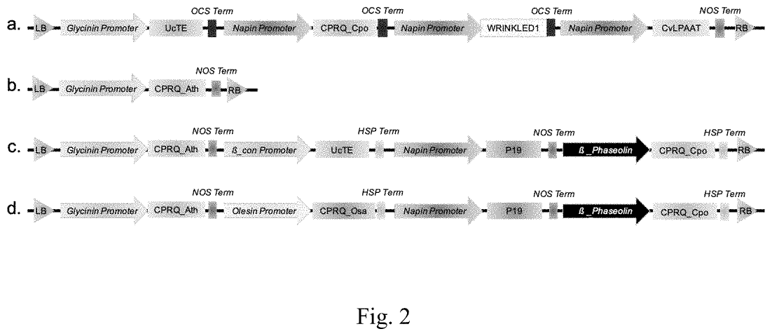

To compare different strategies toward production of high amounts of C₁₂ pheromone precursors in Camelina, four plant expression vectors were constructed (FIG. 2). CPRQ1.0 contained four exogenous genes, which were UcTE controlled by Glycinin, CpCPRQ, AtWRINKLED1 and CvLPAAT controlled by napin (FIG. 2a). CPRQ1.1 contained one exogenous gene CpCPRQ codon optimized for Arabidopsis thaliana (Arabidopsis), controlled by Glycinin (FIG. 2b). CPRQ2.1 contained four exogenous genes controlled by different promoters, including two copies of CpCPRQ with or without codon optimized for Arabidopsis, a UcTE, and a virus silencing suppressor protein P19 (FIG. 2c). Similar to CPRQ2.1, CPRQ2.2 also contained four exogenous genes, which were three copies of CpCPRQ (one without codon optimized, one codon optimized for Arabidopsis, one codon optimized for Oryza sativa) controlled by different promoters, and a P19 (FIG. 2d). Except for CPRQ1.0 which was transformed into wild type Camelina, the other three vectors were transformed into high lauric acid type Camelina.

PCR amplification was performed using the entry clone as template with a pair of degenerate primers, on a Veriti Thermo Cycler, using Phusion Flash High-Fidelity PCR Master Mix (Thermo Scientific™) under conditions as follows: start at 98° C. for 30 s, and 38 cycles at 98° C. for 5 s, 55° C. for 10 s and 72° C. for 50 s, followed by a final extension step at 72° C. for 10 mins. Subsequently, fusion PCR was performed using phusion®Taq (Thermo Scientific™) (Atanassov et al., 2009) to do truncation and gene fusion for gene assembly, the same PCR programs as described before. All genes with promoters and terminators were cloned into the plant expression vector pBinGlyBar (Nguyen et al. 2013), which contained a bar marker gene for Basta selection of transformed plants, by using Multisite Gateway® recombination cloning technology (Invitrogen). The constructed expression clones were confirmed by sequencing.

### Floral Dip Transformation of Camelina by Agrobacterium-Media

The constructed expression vectors were introduced into Agrobacterium tumefaciens strain GV3101 (MP90RK) by electroporation (1700Vmm⁻¹, 5 ms, Eppendorf 2510). The transformed Agrobacterium cells were grown on solid LB medium supplemented with antibiotics 50 mg/L rifampicin, 50 mg/L gentamicin and 50 mg/L spectinomycin after incubating at 30° C. for 36 h. Afterwards, a single clone from each expression clone was incubated in 2 mL liquid LB medium with antibiotics as described above at 30° C. for 36 h. The Agrobacterium solution was then transferred to 30 mL medium for a 36 h incubation, and after that, the solution was transferred to 1 L medium for a 24 h incubation. Subsequently, the 5 weeks old Camelina plants were transformed by the floral dip/vacuum infiltration method as described by Lu and Kang (2008) and Liu et al., 2012. Basta resistant gene was used as a selection marker (Nguyen et al. 2013).

### Fatty Acid Analysis of Seed Oils

Seeds harvested from the floral-dipped plants were sown in soil for Basta selection, and the surviving plants were T1 plants and considered as transformants. T2 seeds were harvested from matured T1 plants for fatty acid analysis. Fatty acids were analyzed as fatty acid methyl esters (FAMEs), which were generated from putative transformants either by grinding 25 pooled T2 seeds (for production analysis of one transformant) or by grinding 15 T2 seeds individually (for variation analysis within one transformant) from each T1 plants in 1 mL 2% H₂SO₄ in methanol in a 4 mL glass vial. After grinding, the samples were incubated for 1 h at 90° C. After cooling, 1 mL water and 1 mL heptane were added and the vial was vortexed. Then, the heptane phase containing the FAMEs was transferred to a new Agilent vial for GC/MS analysis on an Agilent 5975 mass-selective detector, coupled to an Agilent 6890 series gas chromatograph equipped with a polar column (HP-INNOWax, 30 m×0.25 mm, 0.25 μm), and helium was used as carrier gas. The oven temperature was set at 80° C. for 1 min, then increased to 230° C. at a rate of 10° C./min and held for 10 min. Fatty acid compounds were identified by comparison of retention times and mass spectra with those of reference compounds. To determine the position of double bonds in unsaturated fatty acids, DMDS derivatization was performed according to Dunkelblum et al. (1985). The DMDS-adducts were analyzed by GC/MS on a non-polar column (HP-5MS, 30 m×0.25 mm, 0.25 μm) under the following oven temperature program: 80° C. for 2 min, then increased at a rate of 15° C./min to 140° C., and then increased at a rate of 5° C./min to 260° C., and held for 30 min.

### Assembly of Pathways for Moth Pheromone Precursor Biosynthesis and Accumulation in Camelina Seeds

The thioesterase encoded by UcTE taking the laurate plastid acyl carrier protein (ACP) from chain elongation, to form the corresponding lauric (12:0) acid. The free fatty acid is acylated to 12:CoA when transported out of the plastid into the cytosol. The 12:CoA is subsequently converted into corresponding pheromone precursors Z/E9-12:CoA, and E8,E10-12:CoA by the desaturase encoded by CpCPRQ.

### C₁₂ Pheromone Precursors were Produced in Camelina Transformants

GC/MS analysis of C₁₂ to C₁₈ chain length fatty acids showed that wild type Camelina seeds contained the highest amount of linolenic acid (18:3), followed by linoleic acid (18:2) and oleic acid (18:1). In addition, small fractions of lauric acid, myristic acid, and arachidic acid were found in the seeds. In the high lauric acid (12:0) Camelina seeds, the amount of 12:0 was as high as 18:3, and 18:1 was the second most abundant fatty acid.

The C₁₂ pheromone precursors were produced under every strategy in the present study. By analyzing 25 pooled seeds from each transformant, we found that among the nine transformants produced by introduction of gene cassette CPRQ1.0, the most productive Camelina produced 1.7% of E9-12:acid and 0.15% of E8,E10-12:acid from the total fatty acids. The percentage of fatty acids here was calculated as weight percent of total fatty acids. CPRQ1.1 was transformed into high lauric acid Camelina, but the high concentration of 12:0 did not lead to higher production of the unsaturated products. In total, 85 transformants were obtained from CPRQ1.1, of which the best line produced 1.6% of E9-12:acid and 0.15% of E8,E10-12:acid. Similar to CPRQ1.1, the vectors of CPRQ2.1 and CPRQ2.2 were separately transformed into high lauric acid Camelina. In total, we obtained 96 and 30 transformants of CPRQ2.1 and CPRQ2.2, respectively. The results of fatty acids analysis showed that about 83 of CPRQ2.1 transformants lost a lot of the content of 12:0, which dropped from 20% to 3% and no unsaturated pheromone precursor was produced. The other 13 transformants of CPRQ2.1 produced a higher amount of 12:0, increasing up to 31.6%, and produced up to 2.3% of E9-12:acid and 0.20% of E8,E10-12:acid. In the CPRQ2.2 transformants, abundance of E9-12:acid and E8,E10-12:acid in 25 pooled seed was 6.4% and 3.7%, respectively from the most productive plant.

### Comparison of Pheromone Precursors Production between Four Strategies

Comparing the use ratio of 12:0 in CPRQ1.0 transformants to CPRQ1.1 and CPRQ2.1 showed that the CpCPRQ in the former transformants converted a higher percentage of 12:0 into unsaturated pheromone precursors. The CPRQ2.2 transformants converted highest percentage of 12:0 into corresponding pheromone precursors.

Compared to CPRQ1.0 and 1.1, the CPRQ2.1 and 2.2 transformants which contained multiple gene copies and P19 increased the production of pheromone precursors. The most productive Camelina harboring CPRQ2.1 contained up to 2.3% of E9-12:acid and 0.2% of E8,E10-12:acid, which is higher than the best ones in CPRQ1.0 and CPRQ1.1. The Camelina expressing gene cassette of CPRQ2.2 which contained three copies of the Δ9 desaturase CpCPRQ produced much higher E9-12:acid and E8,E10-12:acid than CPRQ2.1 that had two copies of CpCPRQ. In addition, no abnormal plant development was observed among CPRQ2.1 and CPRQ2.2 transformants.

### Increase of Oleic Acid in CPRQ2.1 Transformants

The CPRQ2.1 transformants showed two different fatty acid profile groups. In the first profile group, the 12:0 or 18:3 is the dominant fatty acid, followed by 18:2 and 18:1, which is similar to other transformants (CPRQ1.0, CPRQ1.1, CPRQ2.2), the wild type and the high lauric type. In the second profile group, oleic acid (18:1) is the dominant fatty acid species, which was shown in the 13 of the CPRQ2.1 transformants with a decreased amount of 12:0. Moreover, the ones that lost the 12:0 did not produce any unsaturated pheromone precursors.

The successful production of mono- and di-unsaturated C₁₂ pheromone precursors in Camelina seeds confirmed the “in-planta” function of the Δ9 desaturase derived from C. pomonella. When CpCPRQ was expressed in Camelina it could produce E9-12:acyl from lauric acid as well as a small amount of the corresponding Z isomer. Furthermore, it had the activity to convert the monoene intermediate into E8,E10-12:acid with conjugated double bonds. This gene bifunctionality is consistent with the previous study of Ding and Löfstedt, unpublished using the sf9 insect cell expression system. Our studies substantiate the application of the plant expression system for functional characterization of insect pheromone biosynthetic genes, as well as demonstrated the feasibility of production of diene moth sex pheromone precursors in plant.

We established four different types of transformant lines by using different exogenous gene cassettes. In the regenerated Camelina seeds, the mono-unsaturated E9-12:acid with small amount of (Z)-9-dodecenoic acid (Z9-12:acid) and diunsaturated E8,E10-12:acid were produced in all the four types of transformant lines. Comparing the fatty acid profiles of transformants from CPRQ1.0 and CPRQ1.1 suggests that co-expression with the transcription factor WRINKLED1 from Arabidopsis AtWRI1 and one of the Kennedy pathway gene lysophosphatidic acid acyltransferase from C. viscosissima CvLPAAT can improve the substrate utilization of 12:0. For example, the mean use ratio of 12:0 in CPRQ1.0 was 7.5%, while in CPRQ1.1 transformant it was 2.9%. Also, this implied that the bottleneck of production of pheromone precursors were due to the activity of CpCPRQ, but not the amount of 12:0. Among 85 transformants from CPRQ1.1, containing ca. 20% of 12:0, the most productive plant produced even lower E9-12:acid and same level of E8,E10-12:acid as compared to CPRQ1.0, containing at the most 10% of 12:0 in nine transformants.

Nevertheless, CpCPRQ showed high activity towards E9-12:acid and E8,E10-12:acid production in insect cells (Ding and Löfstedt, unpublished). Why did CpCPRQ show a lower activity in Camelina CPRQ1.0 and 1.1 transformants? One explanation could be the silencing effect of transgenes in Camelina, i.e., small RNAs-guided silencing pathways in plants may have the ability to shut down the exogenous expression (Hagan et al. 2003; Schubert et al. 2004). By expressing two other gene cassettes CPRQ2.1 and CPRQ2.2 in Camelina seeds, we concluded that the low activity of CpCPRQ might indeed be caused by transgene silencing. Both CPRQ2.1 and CPRQ2.2 contained the gene encoding P19 which is a viral silencing suppressor protein (VSP) from Tomato bushy stunt virus (TBSV), was reported to have the ability to suppress the silencing effect of transgenes in previous studies (Naim et al. 2016). Naim et al. 2016 demonstrated that expression of silencing-suppressor protein can protect and enhance stable transgene performance. Here, the expression of CPRQ2.1 and CPRQ2.2 did increase the production of E/Z9-12: acid and E8,E10-12: acid significantly.

Additionally, the VSPs have been widely used together with transgene cassettes to enhance their expression during transient expression experiments. But for the stable transformation the VSP is rarely utilized and explored as they may interfere with endogenous microRNA-regulated process and lead to abnormal plant development (Dunoyer et al. 2004; Fusaro et al. 2012). In the present study, we investigated the use of fully functional P19 expressed only in seeds and controlled by the napin promoter. We demonstrated that stably expressed P19 in Camelina seeds with napin promoter did not cause any abnormal plant development. The germination rate of the most productive seeds from CPRQ2.2 was as high as 95%. This may be due to the native napin promoter driving the seed storage proteins like napin, whose physiological role is to provide the growing seedling with essential nutrients prior to the establishment of the photosynthetic capacity (Rask et al. 1998). Thus, using the napin promoter to control the expression of P19 would not disrupt the small RNA driven regulatory pathways towards the development of vegetative shoot in Camelina (Wong et al. 2011).

Additionally, it was surprising to find that the best tested individual seed from CPRQ2.1 #010 transformant only produced 0.5% of E8,E10-12:acid, while the seed from CPRQ2.2 #005 transformant produced 6.3% of E8,E10-12:acid. This means that one more copy only of CpCPRQ contributed to a more than ten times higher production amount of this di-unsaturated pheromone precursor. Thus, it demonstrated that multiple gene copies are performing excellently in Camelina towards the di-unsaturated pheromone precursors production. Also, we observed a large variation in the production among the different individual seeds, which means that the production of these C₁₂ pheromone precursors in seeds are still unstable and it is also implied that there is a potential to obtain improved Camelina lines producing even higher concentration of the target compounds.

In conclusion, in the present study we investigated the possibility of producing di-unsaturated moth pheromone precursors in transgenic plants. Camelina was genetically modified for production of E8,E10-12:acid via stable integration of gene cassettes using Agrobacterium-mediated floral dip transformation. This is the first report on production of di-unsaturated pheromone precursors in plants. Furthermore, the production amount of 6.3% of E8,E10-12:acid of total fatty acids is quite high. Because the oil content of the Camelina seeds, on a dry weight basis, is typically between 35 to 45% and the yields of Camelina are in a range of 336 to 2240 kg of seeds per hectare (Moser 2010). This means that 7.4 to 63.5 kg (minimum to maximum) of E8,E10-12:acid might be produced by cultivating our best Camelina line. Moreover, in this study we also investigated four strategies for optimization of the plant factory for production of E8,E10-12:acid. We demonstrated that co-expression of the desaturase with P19 and multiple gene copies can increase the production of C₁₂ pheromone precursors significantly. Also, it was confirmed that stably expressing P19 regulated by the seed-specific napin promoter would not cause observable harm of plant development.

### New Platform for Functional Assay of Pheromone Biosynthetic Genes

In this disclosure, a new concept for molecular mechanism exploration of pheromone biosynthesis is provided, which is using N. benthamiana transient expression platform. The idea emerged from the principle of plant factories. Transient expression of various insect genes in plant leaves as factories for pheromone production has been demonstrated for several years (Ding et al. 2014), but utilizing this platform for gene function studies has never been explored. In this study, we confirm that the plant transient expression system is efficient and useful.

Plants were used as a platform for functional characterization of the C. suppressalis biosynthetic gene candidates in order to compare the results to the yeast expression system. The observed functions of these genes in yeast and plant are similar and complementary. For instance, it is interesting that CsupKPSE showed C₁₈>C₁₆ substrate preference when oleic acid was absent in ole1/elo1 yeast. However, in the plant, CsupKPSE shows its preference on C₁₆>C₁₈ because the leaf does not lack oleic acid. The phylogenetic analysis indicated that CsupKPSE belongs to the C₁₆>C₁₈ Glade. This implies that CsupKPSE can adjust its function to produce oleic acid similar to the function of ancestral metabolic desaturase when the nutrition is limited. Therefore, expression of desaturase genes in plant leaves for identification of different functions has the advantage of avoiding the problem of supplemented nutrition inference, especially for Δ9 desaturase identification. A FAR gene CsupFAR2 expressed low activity in yeast, while in plants it shows very high activity. Because the plant lipids are different from yeast, it offers an alternative way for gene function studies. The pgFAR CsupFAR2 showed additional minor activities on polyunsaturated fatty acids of linoleic acid and α-linolenic acid in N. benthamiana, which has never been reported in previous FAR function studies. Moreover, the algae ELO IgalASE1 elongated linoleic acid (18:2) and α-linolenic acid (18:3) to eicosadienoic acid (20:2) and eicosatrienoic acid (20:3) in N. benthamiana, show the same function as reported in yeast S. cerevisiae expression system (33), indicating that heterologous expression of ELO in plant leaves for functional characterization is reliable and feasible.

### New ACO, ELO and FAR Findings

The specific skeleton of an unsaturated fatty acyl chain is generally produced by combination of FAD and ELO or ACO. Among the functionally characterized pheromone biosynthetic genes in L. botrana and C. suppressalis, there were two novel ACO genes Lbo_31670, Lbo_49602 and a first reported ELO gene CsupELO4 showing functions. The former two genes encoding two acyl-CoA oxidases that may produce Z9-12:acid by chain shortening of Z11-14:acid. The later gene encoding an elongase may produce Z13-18:acid, an immediate precursor of a pheromone component in C. suppressalis.

We also functionally characterized a new FAR gene from C. suppressalis, CsupFAR2 that shows very high activity to Z11-16:acid, producing corresponding Z11-16:OH. It also has other minor activity with C₁₆-C₁₈ fatty acids, similar to HarFAR from Helicoverpa armigera (Hagström et al. 2012; 2013), but having more specific activities on Z11-16:acid. The high activity and substrate specificity of CsupFAR2 suggests it to be a valuable candidate for further pheromone production in both yeast and plant factories.

### Increase the Production Amount of Pheromone Compounds—Trichome Specific Promoter

A long-term vision is to produce genetically modified plants that eventually can be used in intercropping as natural dispensers of pheromones and as part of a push-pull (Cook et al. 2007) strategy, providing an innovative and environmentally friendly approach for pest management. Production of a high yield of pheromones in plants by stable transformation is still challenging. With the purpose of producing fatty alcohols or acetates especially for releasing these compounds from the leaf, a trichome specific promoter derived from N. tabacum was cloned and used for FAR and ATF gene expression.

In moths, after the immediate acyl-CoA precursors are produced, FAR can catalyze the reduction of acyl-CoA into fatty alcohols, which either are used as pheromones for some moth species, or in some other moths, converted to corresponding acetates after trans-acetylation by ATFs. Therefore, in order to explore the possibility of releasing pheromone alcohol and acetate in transformed plants, the FAR gene HarFAR and the ATF gene ATF1 was constructed with a trichome specific promoter CYP71D16, respectively, producing the pheromones Z11-16:OH and Z11-16:OAc.

CYP71D16 was confirmed as a trichome-specific promoter leading the downstream gene to be specifically expressed in plant trichomes (Wang et al. 2002). After 3-5 days, the infiltrated N. benthamina plant expressing HarFAR controlled by CYP71D16 promoter produced 70 μg Z11-16:OH in per gram fresh leaf, while the plant expressing HarFAR drived by CaMv35S promoter only produced a tiny amount of Z11-16:OH. The same combination of CpFatB1, AtrΔ11 and HarFAR drived by 35S promoter were also tested in the study of Ding et al. 2014, which reported 18 μg Z11-16:OH in per gram fresh leaf was produced. Also, ATF1 controlled by CYP71D16 expressing in the plant produced much more Z11-16:OAc than 35S. This result suggested that CYP71D16 promoter can increase the pheromone production amount significantly compared to 35S promoter.

### CONCLUSIONS AND PERSPECTIVES

Initially, we characterized several key pheromone biosynthetic genes in the serious pest species, including two beet armyworm Spodoptera species, European grapevine moth L. botrana and the rice stem borer C. suppressalis. These findings will not only help understanding the mechanisms of pheromone biosynthesis, but will also provide many putative candidates for pheromone production in bio-factories.

In the perspective of utilizing results from basic science for application, the second contribution of this disclosure is that we established several stable transgenic plant lines for pheromone precursors production, ranging from the carbon chain length of 12 to 16, either in the leaves or seeds. This is the first report on an extended production of pheromone precursors (C₁₂ and C₁₄) over generations in plants. We confirm that N. benthamiana is a suitable platform for stable production of C₁₆ pheromone precursors. We also established productive Camelina line for di-unsaturated C₁₂ pheromone precursor production. Up to 6.3% of E8,E10-12:acid of total fatty acids was achieved. Because the oil content of the Camelina seeds, on a dry weight basis, is typically between 35 to 45% and the Camelina yields anywhere are from 336 to 2240 kg of seeds per hectare, which means 7.4 to 63.5 kg (minimum to maximum) of E8,E10-12:acid might be produced by cultivating our best Camelina line.

A further aim is to enable stable production of final pheromone components in bio-factories, which remains to be further explored. First, increasing the pheromone precursor production to provide more substrate for pheromone biosynthesis is one of the possible approaches. How to increase the precursor production, for instance by elevating the TAG accumulation in plant cells, needs further efforts. Our long-term goal is to design the “tailor-made” production of any moth pheromone in stably transformed plants. However, much remains unknown about the molecular mechanisms underlying the pheromone biosynthesis. The identification of genes encoding pheromone biosynthesis enzymes is a crucial step in ensuring the compatibility of bio-factories. Prior to this disclosure, only FAD and FAR genes have been functionally characterized, but we now have found several functional genes encoding ACOs and ELO that were involved in pheromone biosynthesis. We also developed the plant platform for insect gene functional characterization, contributing an efficient way for further fatty acid metabolic and pheromone biosynthesis studies.

Ultimately, plant factory for pheromone production could be used in a “push-pull” strategy for pest control, which is using pheromones that act to make the protected resource unattractive to the pests (push) while luring them toward an attractive source (pull) from where the pests are subsequently removed. This requires that the plants can release pheromones into the atmosphere which is challenging. We intended to accomplish this by using a trichome-specific promoter CYP71D16 to drive pheromone biosynthetic genes. It was surprising to find that this strategy increased the pheromone production amount quite substantially.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

### REFERENCES

Agaba M., Tocher D. R., Dickson C. A., Dick J. R., and Teale A. J. (2004). Zebrafish cDNA encoding multifunctional fatty acid elongase involved in production of eicosapentaenoic (20: 5n-3) and docosahexaenoic (22: 6n-3) acids. Marine Biotechnol. 6, 251-261.
Anderson C. M., and Stahl A. (2013). SLC27 fatty acid transport proteins. Mol. Aspects. MED. 34, 516-528.
Ando T., Inomata S. and Yamamoto M. (2004). Lepidopteran Sex Pheromones. Top Curr. Chem. 239, 51-96.
Arsequell G., Fabrias G. and Camps F. (1990). Sex pheromone biosynthesis in the processionary moth Thaumetopoea pityocampa by delta-13 desaturation. Arch. Insect. Biochem. Physiol. 14, 47-56.
Atanassov I. I. , Atanassov I. I., Etchells J., and Turner, S. R. (2009). A simple, flexible and efficient PCR-fusion/Gateway cloning procedure for gene fusion, site-directed mutagenesis, short sequence insertion and domain deletions and swaps. Plant methods. 5, 14.
Bansal S., and Durrett T. P. (2016). Camelina sativa: an ideal platform for the metabolic engineering and field production of industrial lipids. Biochimie. 1, 9-16.
Beale M. H., Birkett M. A., Bruce T. J., Chamberlain K., Field L. M., et al. (2006). Aphid alarm pheromone produced by transgenic plants affects aphid and parasitoid behavior. Proc. Nat. Acad. Sci. 103, 10509-10513.
Behrouzian B. and Buist P. H. (2002). Fatty acid desaturation: variations on an oxidative theme. Curr. Opin. Chem. Biol. 6, 577-582.
Bjostad L. B., and Roelofs W. L. (1983). Sex Pheromone Biosynthesis in Trichoplusia ni: Key Steps Involve Delta-11 Desaturation and Chain-Shortening. Science 220, 1387-1389.
Bjostad L. B., and Roelofs W. L. (1984). Sex pheromone biosynthetic precursors in Bombyx mori. Insect biochem. 14, 275-278.
Bjostad L. B., and Roelofs, W. L. (1981). Sex pheromone biosynthesis from radiolabeled fatty acids in the redbanded leafroller moth. J. Biol. Chem. 256, 7936-7940.
Black P. N., and DiRusso C. C. (2007). Vectorial acylation: linking fatty acid transport and activation to metabolic trafficking. In Fatty Acids and Lipotoxicity in Obesity and Diabetes: Novartis Foundation Symposium, John Wiley and Sons, Ltd. 286, 127-141.
Boehm R. (2007). Bioproduction of therapeutic proteins in the 21st century and the role of plants and plant cells as production platforms. Ann. NY. Acad. Sci. 1102, 121-134.
Brittain C., and Potts S. G. (2011). The potential impacts of insecticides on the life-history traits of bees and the consequences for pollination. Basic Appl. Ecol. 12, 321-331.
Buček A., Matoušková P., Vogel H., Šebesta P., Jahn U., et al. (2015). Evolution of moth sex pheromone composition by a single amino acid substitution in a fatty acid desaturase. Proc. Nat. Acad. Sci. 112, 12586-12591.
Burton M., Rose T. M., Faergeman N. J., and Knudsen J. (2005). Evolution of the acyl-CoA binding protein (ACBP). Biochem J. 392, 299-307
Butenandt V. A. (1959). Uber den sexsual-lockstoff des seidenspinners Bombyx mori. Reindarstellung and konstitution. Z. Naturforschg. B. 14, 283.
Cahoon E. B., Shockey J. M., Dietrich C. R., Gidda S. K., Mullen, R. T., et al. (2007). Engineering oilseeds for sustainable production of industrial and nutritional feedstocks: solving bottlenecks in fatty acid flux. Curr. Opin. Plant Biol. 10, 236-244.
Centini F., Masti A., and Comparini I. B. (1996). Quantitative and qualitative analysis of MDMA, MDEA, MA and amphetamine in urine by head-space/solid phase micro-extraction (SPME) and GC/MS. Forensic Sci. Int. 83, 161-166.
Cernac A., and Benning C. (2004). WRINKLED1 encodes an AP2/EREB domain protein involved in the control of storage compound biosynthesis in Arabidopsis. Plant J. 40, 575-585.
Chertemps T., Duportets L., Labeur C., Ueda R., Takahashi K., et al. (2007). A female-biased expressed elongase involved in long-chain hydrocarbon biosynthesis and courtship behavior in Drosophila melanogaster. Proc. Nat. Acad. Sci. 104, 4273-4278.
Chertemps T., Duportets L., Labeur C., and Thomas, C. W. (2005). A new elongase selectively expressed in Drosophila male reproductive system. Biochem. Biophys. Res. Commun. 333, 1066-1072
Christie W. W. (1998). Gas chromatography-mass spectrometry methods for structural analysis of fatty acids. Lipids 33, 343-353.
Clemente T. (2006). Nicotiana (Nicotiana tobaccum, Nicotiana benthamiana). In Agrobacterium protocols. Humana Press. 143-154.
Clinkenbeard K. D., Sugiyama T., Moss J., Reed W. D., and Lane, M. D. (1973). Molecular and catalytic properties of cytosolic acetoacetyl coenzyme A thiolase from avian liver. J. Biol. Chem. 248, 2275-2284.
Cloney R. (2016). Synthetic biology: Automating genetic circuit design. Nat. Rev. Genetics 17, 314.
Conner W. E., Eisner T., Vander Meer R. K., Guerrero A., Ghiringelli D., et al. (1980). Sex attractant of an arctiid moth (Utetheisa ornatrix): a pulsed chemical signal. Behav. Ecol. Sociobiol. 7, 55-63.
Cook S. M., Khan Z. R., and Pickett J. A. (2007). The use of push-pull strategies in integrated pest management. Annu. Rev. Entomol. 52, 375-400.
Dehesh K., Edwards P., Hayes T., Cranmer A. M., and Fillatti J. (1996a). Two novel thioesterases are key determinants of the bimodal distribution of acyl chain length of Cuphea palustris seed oil. Plant Physiol. 110, 203-210.
Ding B. J., Carraher C., and Löfstedt C. (2016). Sequence variation determining stereochemistry of a Δ11 desaturase active in moth sex pheromone biosynthesis. Insect Biochem. Mol. Biol. 74, 68-75.
Ding B. J., Hofvander P., Wang H. L., Durrett T. P., Stymne S., et al. (2014). A plant factory for moth pheromone production. Nat. commun. 5, 3353.
Ding B. J., Liénard M. A., Wang H. L., Zhao C. H., and Löfstedt C. (2011). Terminal fatty-acyl-CoA desaturase involved in sex pheromone biosynthesis in the winter moth (Operophtera brumata). Insect Biochem. Mol. Biol. 41, 715-722.
DiRusso C. C., Li H., Darwis D., Watkins P. A., Berger J., et al. (2005). Comparative biochemical studies of the murine fatty acid transport proteins (FATP) expressed in yeast. J. Biol. Chem. 280, 16829-16837.
Duke S. O. (1994). Glandular trichomes-a focal point of chemical and structural interactions. Intern. J. Plant Sci. 155, 617-620.
Dunkelblum E., Tan S. H., and Silk P. J. (1985). Double-bond location in monounsaturated fatty acids by dimethyl disulfide derivatization and mass spectrometry: Application to analysis of fatty acids in pheromone glands of four lepidoptera. J. Chem. Ecol. 11, 265-277.
Dyer J. M., Stymne S., Green A. G., and Carlsson A. S. (2008). High-value oils from plants. Plant J. 54, 640-655.
Eccleston V. S., and Ohlrogge J. B. (1998). Expression of lauroyl-acyl carrier protein thioesterase in Brassica napus seeds induces pathways for both fatty acid oxidation and biosynthesis and implies a set point for triacylglycerol accumulation. Plant Cell 10, 613-621.
Færgeman N. J., Wadum M., Feddersen S., Burton M., Kragelund B. B., et al. (2007). Acyl-CoA binding proteins; structural and functional conservation over 2000 MYA. Mol. Cellular Biochem. 299, 55-65.
Filichkin S. A., Slabaugh M. B., and Knapp S. J. (2006). New FATB thioesterases from a high laurate Cuphea species: functional and complementation analyses. Eur. J. Lipid Sci. Tech. 108, 979-990.
Foster S. P. (2005). Lipid analysis of the sex pheromone gland of the moth Heliothis virescens. Arch. Insect. Biochem. Physiol. 59, 80-90.
Foster S. P., and Roelofs W. L. (1988). Sex pheromone biosynthesis in the leafroller moth Planotortrix excessana by Δ10 desaturation. Arch. Insect. Biochem. Physiol. 8, 1-9.
Foster S. P., and Roelofs W. L. (1996). Sex pheromone biosynthesis in the tortricid moth, Ctenopseustis herana (Felder and Rogenhofer). Arch. Insect. Biochem. Physiol. 33, 135-147.
Ghosh S. K., Bhattacharjee A., Jha J. K., Mondal A. K., Maiti M. K., et al. (2007). Characterization and cloning of a stearoyl/oleoyl specific fatty acyl-acyl carrier protein thioesterase from the seeds of Madhuca longifolia (latifolia). Plant Physiol. Biochem. 45, 887-897.
Gibbs W. W. (2004). Synthetic life. Sci. Am. 290, 74-81
Graham S. A. (1998). Revision of Cuphea section Diploptychia (Lythraceae). Syst. Bot. 53, 1-96.
Graham S. A., and Kleiman R. (1992). Composition of seed oils in some Latin American Cuphea (Lythraceae). Ind. Crop. Prod. 1, 31-34.
Green C. D., and Ozguden-Akkoc C. G., Wang Y., Jump D. B., and Olson L. K. (2010). Role of fatty acid elongases in determination of de novo synthesized monounsaturated fatty acid species. J. Lipid Res. 51, 1871-1877.
Grimberg A., Carlsson A. S., Marttila S., Bhalerao R., and Hofvander P., 2015. Transciptional transitions in Nicotiana benthamiana leaves upon induction of oil synthesis by WRINKLED1 homologs from diverse species and tissues. BMC Plant Biol. 15, 192.
Guo Z., and Wagner G. J. (1995). Biosynthesis of labdenediol and sclareol in cell-free extracts from trichomes of Nicotiana glutinosa. Planta 197, 627-632.
Hagström Å. K., Albre J., Tooman L. K., Thirmawithana A. H., Corcoran J., et al. (2014). A novel fatty acyl desaturase from the pheromone glands of Ctenopseustis obliquana and C. herana with specific Z5-desaturase activity on myristic acid. J. Chem. Ecol. 40, 63-70.
Hagström Å. K., Liénard M. A., Groot A. T., Hedenström E., and Löfstedt C. (2012). Semi-selective fatty acyl reductases from four heliothine moths influence the specific pheromone composition. PLoS One, 7, e37230.
Hagström Å. K., Wang H. L., Liénard M. A., Lassance J. M., Johansson, T., et al. (2013). A moth pheromone brewery: production of (Z)-11-hexadecenol by heterologous co-expression of two biosynthetic genes from a noctuid moth in a yeast cell factory. Microb. Cell Fact. 12, 125.
Hao G., Liu W., O'Connor M., and Roelofs W. L. (2002). Acyl-CoA Z9-and Z10-desaturase genes from a New Zealand leafroller moth species, Planotortrix octo. Insect Biochem. Mol. Biol. 32, 961-966.
Hashimoto K., Yoshizawa A. C., Okuda S., Kuma K., Goto S., et al. (2008). The repertoire of desaturases and elongases reveals fatty acid variations in 56 eukaryotic genomes. J. Lipid Res. 49, 183-191.
Hawkins D. J., and Kridl J. C. (1998) Characterization of acyl-ACP thioesterases of mangosteen (Garcinia mangostana) seed and high levels of stearate production in transgenic canola. Plant J. 13, 743-752.
Heppner J. B. (1991). Faunal regions and the diversity of Lepidoptera. Tropical Lepidoptera 2, 1-85.
Horsch R. B., Rogers S. G., and Fraley R. T. (1985) Transgenic plants, Cold Spring Harb. Symp. Quant. Biol. 50, 433-437.
Iskandarov U., Kim H. J., and Cahoon E. B. (2014). Camelina: an emerging oilseed platform for advanced biofuels and bio-based materials. In Plants and bioenergy, McCann M.C., Ed, Springer, New York Press, pp, 131-140.
Jeong H., Néda Z., and Barabási A. L. (2003). Measuring preferential attachment in evolving networks. Europhys. Lett. 61, 567.
Jones A., Davies H. M., and Voelker T. A. (1995). Palmitoyl-acyl carrier protein (ACP) thioesterase and the evolutionary origin of plant acyl-ACP thioesterases. Plant Cell 7,359-371.
Jump D. B. (2009). “Mammalian fatty acid elongases” In Lipidomics, Totowa N.J., Ed, Humana Press, pp, 375-389.
Jurenka R. (2004). The Chemistry of Pheromones and Other Semiochemicals I. In Insect pheromone biosynthesis. Springer Berlin Heidelberg Press, pp, 97-132.
Jurenka R. (1997). Biosynthetic pathway for producing the sex pheromone component (Z,E)-9,12-tetradecadienyl acetate in moths involves a Δ12 desaturase. Cell. Mol. Life. Sci. 53, 501-505.
Kandra L., and Wagner G. J. (1988). Studies of the site and mode of biosynthesis of tobacco trichome exudate components. Arch. Biochem. Biophysic. 265, 425-432.
Karg S. R., and Kallio P. T. (2009). The production of biopharmaceuticals in plant systems. Biotechnol. Adv. 27, 879-894.
Katzen F. (2007). Gateway® recombinational cloning: a biological operating system. Expert. Opin. Drug Dis. 2, 571-589.
Kim H. J., Silva J. E., Vu H. S., Mockaitis K., Nam J. W., et al. (2015a). Toward production of jet fuel functionality in oilseeds: identification of FatB acyl-acyl carrier protein thioesterases and evaluation of combinatorial expression strategies in Camelina seeds. J. Exp. Bot. 66, 4251-4265.
Knaut J., and Richtler H. (1985). Trends in industrial uses of palm and lauric oils. J. Am. Oil Chem.' Soc. 62, 317-327.
Knipple D. C., Rosenfield C. L., Miller S. J., Liu W., and Tang J., et al. (1998). Cloning and functional expression of a cDNA encoding a pheromone gland-specific acyl-CoA Δ11-desaturase of the cabbage looper moth, Trichoplusia ni. Proc. Nat. Acad. Sci. 95, 15287-15292.
Knipple D. C., Rosenfield C. L., Nielsen R., You K. M., and Jeong S. E. (2002). Evolution of the integral membrane desaturase gene family in moths and flies. Genetics 162, 1737-1752.
Knutzon D. S., Bleibaum J. L., Nelsen J., Kridl J. C. and Thompson G. A. (1992) Isolation and characterization of 2 safflower oleoyl-acyl carrier protein thioesterase cDNA clones. Plant Physiol. 100, 1751-1758.
Knutzon D. S., Hayes T. R., Wyrick A., Xiong H., Davies H. M., et al. (1999). Lysophosphatidic acid acyltransferase from coconut endosperm mediates the insertion of laurate at the sn-2 position of triacylglycerols in lauric rapeseed oil and can increase total laurate levels. Plant Physiol. 120, 739-746.
Lamprecht I., Schmolz E., and Schricker B. (2008). Pheromones in the life of insects. Eur. Biophys. J. 37, 1253-1260.
Lassance J. M., Groot A. T., Liénard M. A., Antony B., Borgwardt C., et al. (2010). Allelic variation in a fatty-acyl reductase gene causes divergence in moth sex pheromones. Nature 466, 486.
Leonard J. M., Slabaugh M. B., and Knapp S. J. (1997). Cuphea wrightii thioesterases have unexpected broad specificities on saturated fatty acids. Plant Mol. Biol. 34, 669-679.
Li-Beisson Y., Shorrosh B., Beisson F., Andersson M. X., Arondel V., et al. (2013). Acyl-lipid metabolism. Arabidopsis Book 11, e0161.
Liénard D., Sourrouille C., Gomord V., and Faye L. (2007). Pharming and transgenic plants. Biotech. Ann. Rev. 13, 115-147.
Liénard M. A., Hagström Å. K., Lassance J. M., and Löfstedt C. (2010). Evolution of multicomponent pheromone signals in small ermine moths involves a single fatty-acyl reductase gene. Proc. Nat. Acad. Sci. 107, 10955-10960.
Liénard M. A., Lassance J. M., Wang H. L., Zhao C. H., Piškur J., et al. (2010). Elucidation of the sex-pheromone biosynthesis producing 5, 7-dodecadienes in Dendrolimus punctatus (Lepidoptera: Lasiocampidae) reveals Δ11-and Δ9-desaturases with unusual catalytic properties. Insect Biochem. Mol. Biol. 40, 440-452.
Liu W., Jiao H., Murray N. C., O'Connor M., and Roelofs W. L. (2002). Gene characterized for membrane desaturase that produces (E)-11 isomers of mono-and diunsaturated fatty acids. Proc. Nat. Acad. Sci. 99, 620-624.
Liu W., Jiao H., O'Connor, M., and Roelofs W. L. (2002). Moth desaturase characterized that produces both Z and E isomers of Δ11-tetradecenoic acids. Insect Biochem. Mol. Biol. 32, 1489-1495.
Liu W., Ma P. W. K., Marsella-Herrick P., Rosenfield C. L., Knipple D. C., et al. (1999) Cloning and functional expression of a cDNA encoding a metabolic acyl-CoA Delta 9-desaturase of the cabbage looper moth, Trichoplusia ni. Insect Biochem. Mol. Biol. 29, 435-43.
Liu W., Rooney A. P., Xue B., and Roelofs W. L. (2004). Desaturases from the spotted fireworm moth (Choristoneura parallela) shed light on the evolutionary origins of novel moth sex pheromone desaturases. Gene 342, 303-311.
Löfstedt C., and Bengtsson M. (1988). Sex pheromone biosynthesis of (E,E)-8,10-dodecadienol in codling moth Cydia pomonella involves E9 desaturation. J. Chem. Ecol. 14, 903-915.
Löfstedt C., Hansson B. S., Petersson E., Valeur P., and Richards A. (1994). Pheromonal secretions from glands on the 5th abdominal sternite of hydropsychid and rhyacophilid caddisflies (Trichoptera). J. Chem. Ecol. 20, 153-170.
Löfstedt C., and Kozlov M. (1997). A phylogenetic analysis of pheromone communication in primitive moths., In Insect pheromone research, Boston, M.A. Ed. Springer Press, pp, 473-489.
Löfstedt C., Wahlberg N., and Millar J. M. (2016). Evolutionary patterns of pheromone diversity in Lepidoptera. In Pheromone communication in moths: evolution, behavior and application, Allison J.D. Ed. University of California Press, pp, 43-82.
Lu C., Kang J. (2008) Generation of transgenic plants of a potential oilseed crop Camelina sativa by Agrobacterium-mediated transformation. Plant Cell Rep. 27, 273-278.
Ma J. K., Chikwamba R., Sparrow P., Fischer R., Mahoney R., et al. (2005). Plant-derived pharmaceuticals—the road forward. Trends Plant Sci. 10, 580-585.
Ma P. W., Ramaswamy S. B., Blomquist G. J., and Vogt R. G. (2003). Biology and ultrastructure of sex pheromone-producing tissue. In Insect pheromone biochemistry and molecular biology, Blomquist G. Ed, Academic Press, pp, 19-51.
Mandal M. N. A., Santha I. M., Lodha M. L., and Mehta S. L. (2000). Cloning of acyl-acyl carrier protein (ACP) thioesterase gene from Brassica juncea. Biochem. Soc. Trans. 28, 967-969.
Martinez T., Fabrias G., and Camps F. (1990). Sex pheromone biosynthetic pathway in Spodoptera littoralis and its activation by a neurohormone. J. Biol. Chem. 265, 1381-1387.
Matoušková P., Pichová I., and Svatoš A. (2007). Functional characterization of a desaturase from the tobacco hornworm moth (Manduca sexta) with bifunctional Z11-and 10, 12-desaturase activity. Insect Biochem. Mol. Biol. 37, 601-610.
Mett V., Farrance C. E., Green B. J., and Yusibov V. (2008). Plants as biofactories. Biologicals 36, 354-358.
Michaelis S., and Herskowitz I. (1988). The a-factor pheromone of Saccharomyces cerevisiae is essential for mating. Mol. Cellu. Biol. 8, 1309-1318.
Mogensen I. B., Schulenberg H., Hansen H. O., Spener F., and Knudsen J. (1987). A novel acyl-CoA-binding protein from bovine liver. Effect on fatty acid synthesis. Biochemical J. 241, 189-192.
Moser B. R. (2010). Camelina (Camelina sativa L.) oil as a biofuel's feedstock: Golden opportunity or false hope? Lipid Technol. 22, 270-273.
Moto K. I., Suzuki M. G., Hull J. J., Kurata R., Takahashi S., et al. (2004). Involvement of a bifunctional fatty-acyl desaturase in the biosynthesis of the silkmoth, Bombyx mori, sex pheromone. Proc. Nat. Acad. Sci. 101, 8631-8636.
Moto K. I., Yoshiga T., Yamamoto M., Takahashi S., Okano, K., et al. (2003). Pheromone gland-specific fatty-acyl reductase of the silkmoth, Bombyx mori. Proc. Nat. Acad. Sci. 100, 9156-9161.
Murashige T., and Skoog F. (1962). A revised medium for rapid growth and bio assays with tobacco tissue cultures. Physiol. Plant. 15, 473-497.
Nesšnerová P., Šebek P., Macek T., and Svatoš A. (2004). First semi-synthetic preparation of sex pheromones. Green Chem. 6, 305-307.
Nguyen H. T., Silva J. E., Podicheti R., Macrander J., Yang W., et al. (2013). Camelina seed transcriptome: a tool for meal and oil improvement and translational research. Plant Biotech. J. 11, 759-769.
Oh C. S., Toke D. A., Mandala S., and Martin C. E. (1997). ELO2 and ELO3, Homologues of the Saccharomyces cerevisiae ELO1 Gene, Function in Fatty Acid Elongation and Are Required for Sphingolipid Formation. J. Biol. Chem. 272, 17376-17384
Ohlrogge J. B., Kuhn D. N., and Stumpf P. K. (1979). Subcellular localization of acyl carrier protein in leaf protoplasts of Spinacia oleracea. Proc. Nat. Acad. Sci. 76, 1194-1198.
Othman A., Lazarus C., Fraser T. and Stobart K. (2000). Cloning of a palmitoyl-acyl carrier protein thioesterase from oil palm. Biochem. Soc. Trans. 28, 619-622.
Percy-Cunningham J. E., and MacDonald J. A. (1987). Biology and Ultrastructure of Sex Pheromone-Producing Glands. In Pheromone biochemistry, Prestwich G.D. Ed, Academic Press, pp, 27-75.
Pollard M. R., Anderson L., Fan C., Hawkins D. J., and Davies H. M. (1991). A specific acyl-ACP thioesterase implicated in medium-chain fatty acid production in immature cotyledons of Umbellularia californica. Arch. Biochem. Biophy. 284, 306-312.
Raguso R. A., and Pellmyr O. (1998). Dynamic headspace analysis of floral volatiles: a comparison of methods. Oikos 81, 238-254.
Raina A. K., Wergin W. P., Murphy C. A., and Erbe E. F. (2000). Structural organization of the sex pheromone gland in Helicoverpa zea in relation to pheromone production and release. Arthropod Struct. Dev. 29, 343-353.
Rasmussen L. E. L., Lee T. D., Zhang A., Roelofs W. L., and Daves G. D. (1997). Purification, identification, concentration and bioactivity of (Z)-7-dodecen-1-yl acetate: sex pheromone of the female Asian elephant, Elephas maximus. Chem. Senses 22, 417-437.
Reddy G. V. P., and Guerrero A. (2010). New pheromones and insect control strategies. In Vitamins and Hormones, Massachusetts L.G. Ed. Elsevier Press, pp, 493-519.
Reynolds K. B., Taylor M. C., Cullerne D. P., Blanchard C. L., Wood C. C., et al. (2017). A reconfigured Kennedy pathway which promotes efficient accumulation of medium-chain fatty acids in leaf oils. Plant Biotechnol. J. 15, 1397-1408.
Rodriguez S., Hao G., Liu W., Piña B., Rooney A., et al. (2004). Expression and evolution of Δ9 and Δ11 desaturase genes in the moth Spodoptera littoralis. Insect Biochem. Mol. Biol. 34, 1315-1328
Rodriguez-Rodriguez M. F., Salas J. J., Garces R., and Martinez-Force E. (2014). Acyl-ACP thioesterases from Camelina sativa: cloning, enzymatic characterization and implication in seed oil fatty acid composition. Phytochemistry 107, 7-15.
Raclot T. (1997). Selective mobilization of fatty acids from white fat cells: evidence for a relationship to the polarity of triacylglycerols. Biochemical J. 322, 483-489.
Roelofs W. L., Liu W., Hao G., Jiao H., Rooney A. P., et al. (2002). Evolution of moth sex pheromones via ancestral genes. Proc. Nat. Acad. Sci. 99, 13621-13626.
Rosenfield C. L., You K. M., Marsella-Herrick P., Roelofs W. L., and Knipple D. C. (2001). Structural and functional conservation and divergence among acyl-CoA desaturases of two noctuid species, the corn earworm, Helicoverpa zea, and the cabbage looper, Trichoplusia ni. Insect Biochem. Mol. Biol. 31, 949-964.
Salas J. J., and Ohlrogge J. B. (2002) Characterization of substrate specificity of plant FatA and FatB acyl-ACP thioesterases. Arch. Biochem. Biophys. 403, 25-34.
Sanchez-Garcia A., Moreno-Perez A. J., Muro-Pastor A. M., Salas J. J., Garces R. et al. (2010) Acyl-ACP thioesterases from castor (Ricinus communis L.): an enzymatic system appropriate for high rates of oil synthesis and accumulation. Phytochemistry 71, 860-869.
Schultz D. J., and Ohlrogge J. B. (2001). Metabolic engineering of fatty acid biosynthesis. In Lipid Biotech. Kuo T.M. Ed, Marcel Dekker Inc.
Schuster S. C. (2007). Next-generation sequencing transforms today's biology. Nat. Methods 5, 16.
Serra M., Piña B., Abad J. L., Camps F., and Fabrics G. (2007). A multifunctional desaturase involved in the biosynthesis of the processionary moth sex pheromone. Proc. Nat. Acad. Sci. 104, 16444-16449.
Serra M., Piña B., Bujons J., Camps F., and Fabriàs G. (2006). Biosynthesis of 10, 12-dienoic fatty acids by a bifunctional Δ11 desaturase in Spodoptera littoralis. Insect Biochem. Mol. Biol. 36, 634-641.
Serrano-Vega M. J., Garces R., and Martinez-Force E. (2005) Cloning, characterization and structural model of a FatA-type thioesterase from sunflower seeds (Helianthus annuus L.). Planta 221, 868-880.
Shanklin J., Whittle E., and Fox B. G. (1994). Eight histidine residues are catalytically essential in a membrane-associated iron enzyme, stearoyl-CoA desaturase, and are conserved in alkane hydroxylase and xylane monooxygenase, Biochemistry 33, 12787-12794.
Simmons G. S., Suckling D. M., Carpenter J. E., Addison M. F., Dyck V. A., et al. (2010). Improved quality management to enhance the efficacy of the sterile insect technique for lepidopteran pests. J. Appl. Entomol. 134, 261-273.
Slabaugh M. B., Leonard J. M., and Knapp S. J. (1998). Condensing enzymes from Cuphea wrightii associated with medium chain fatty acid biosynthesis. Plant J. 13, 611-620.
Stahl S. S. (2004). Palladium oxidase catalysis: selective oxidation of organic chemicals by direct dioxygen-coupled turnover. Angew. Chemie. Intern. Ed. 43, 3400-3420.
Strandh M., Johansson T., Ahrén D., and Löfstedt C. (2008). Transcriptional analysis of the pheromone gland of the turnip moth, Agrotis segetum (Noctuidae), reveals candidate genes involved in pheromone production. Insect. Mol. Biol. 17, 73-85.
Ternes P., Franke S., Zahringer U., Sperling P., and Heinz E. (2002). Identification and characterization of a sphingolipid delta 4-desaturase family. J. Biol. Chem. 277, 25512-25518.
Thelen J. J., and Ohlrogge J. B. (2002). Metabolic engineering of fatty acid biosynthesis in plants. Metab. Eng. 4, 12-21.
Tjellström H., Strawsine M., Silva J., Cahoon E. B., and Ohlrogge J. B. (2013). Disruption of plastid acyl:acyl carrier protein synthetases increases medium chain fatty acid accumulation in seeds of transgenic Arabidopsis. FEBS Letters 587, 936-942.
Tocher D. R., Leaver M. J., and Hodgson P. A. (1998). Recent advances in the biochemistry and molecular biology of fatty acyl desaturases. Prog. Lipid Res. 37,73-117.
Toke D. A., and Martin C. E. (1996). Isolation and characterization of a gene affecting fatty acid elongation in Saccharomyces cerevisiae. J. Biol. Chem. 271, 18413-18422.
Tupec M., Buček A., Valterová I., and Pichová I. (2017). Biotechnological potential of insect fatty acid-modifying enzymes. Zeitschrift Für Naturforschung C. 72, 387-403.
Tvrdik P., Westerberg R., Silve S., Asadi A., Jakobsson A., et al. (2000). Role of a new mammalian gene family in the biosynthesis of very long chain fatty acids and sphingolipids. J. Cell Biol. 149, 707-718.
van Nieukerken E. J., Kaila L., Kitching I. J., Kristensen N. P., Lees D. C., et al. (2011). Order Lepidoptera Linnaeus, 1758. Zootaxa, 3148, 212-221.
Vanhercke T., El Tahchy A., Liu Q., Zhou X. R., Shrestha P., et al. (2014b). Metabolic engineering of biomass for high energy density: oilseed-like triacylglycerol yields from plant leaves. Plant Biotechnol. J. 12, 231-239.
Visser J. H., and Thiery D. (1986). Effects of feeding experience on the odour-conditioned anemotaxes of Colorado potato beetles. Entomol. Exp Appl. 42, 198-200.
Voelker T., and Kinney A. J. (2001). Variations in the biosynthesis of seed-storage lipids. Ann. Rev. Plant Biol. 52, 335-361.
Voelker T. A., Hayes T. R., Cranmer A. M., Turner J. C., and Davies H. M. (1996). Genetic engineering of a quantitative trait: Metabolic and genetic parameters influencing the accumulation of laurate in rapeseed. Plant J. 9, 229-241.
Voelker T. A., Worrell A. C., Anderson L., Bleibaum J., Fan C., et al. (1992). Fatty acid biosynthesis redirected to medium chains in transgenic oilseed plants. Science 257, 72-74.
Voinnet O., Rivas S., Mestre P., and Baulcombe D. (2003). An enhanced transient expression system in plants based on suppression of gene silencing by the p19 protein of tomato bushy stunt virus. Plant J. 33, 949-956.
Wang E. M., Gan S. S., and Wagner G. J. (2002). Isolation and characterization of the CYP71D16 trichome-specific promoter from Nicotiana tabacum L. J. Exp. Bot. 53, 1891-1897.
Wang H. L., Liénard M. A., Zhao C. H., Wang C. Z., and Löfstedt C. (2010). Neofunctionalization in an ancestral insect desaturase lineage led to rare Δ6 pheromone signals in the Chinese tussah silkworm. Insect Biochem. Mol. Biol. 40, 742-751.
Weatherston I., and Stewart R. (2002). Regulatory issues in the commercial development of pheromones and other semiochemicals. IOBC wprs Bulletin, 25, 1-10.
Wyatt T. D. (2003). Pheromones and animal behaviour: communication by smell and taste. Cambridge University Press.
Xia Y. H., Zhang Y. N., Ding B. J., Wang H. L., and Löfstedt C. (2019). Multi-Functional Desaturases in Two Spodoptera Moths with Δ11 and Δ12 Desaturation Activities. J. Chem. Ecol. 45, 378-387.
Xia Y. H., Zhang Y. N., Hou X. Q., Li F., and Dong S. L. (2015). Large number of putative chemoreception and pheromone biosynthesis genes revealed by analyzing transcriptome from ovipositor-pheromone glands of Chilo suppressalis. Sci. Rep. 5, 7888.
Yamaoka R., Taniguchi Y., and Hayashiya K. (1984). Bombykol biosynthesis from deuterium-labeled (Z)-11-hexadecenoic acid. Experientia 40, 80-81.
Young M. D., Wakefield M. J., Smyth G. K., and Oshlack A. (2010). Gene ontology analysis for RNA-seq: accounting for selection bias. Genome Biol. 11, 14.
Zale J., Jung J. H., Kim J. Y., Pathak B., Karan R., et al. (2016). Metabolic engineering of sugarcane to accumulate energy-dense triacylglycerols in vegetative biomass. Plant Biotechnol. J. 14, 661-669.
Zhao C., Löfstedt C., and Wang X. (1990). Sex pheromone biosynthesis in the Asian corn borer Ostrinia furnicalis (II): biosynthesis of (E)- and (Z)-12-tetradecenyl acetate involves Δ14 desaturation. Arch. Insect. Biochem. Physiol. 15, 57-65.

What is claimed is:

1. A genetically modified plant having incorporated into the genome a heterologous gene encoding at least one silencing suppressor protein and at least one enzyme selected from the group consisting of a fatty acyl desaturase, a fatty acyl elongase, a fatty acyl reductase, and an acyl-CoA oxidase, wherein the plant produces at least one insect pheromone precursor.

2. The genetically modified plant of claim 1, wherein the at least one silencing suppressor protein is P19.

3. The genetically modified plant of claim 1, wherein the heterologous gene includes multiple copies of the same enzyme.

4. The genetically modified plant of claim 3, wherein the heterologous gene includes three copies of the same enzyme.

5. The genetically modified plant of claim 1, wherein the at least one enzyme is a fatty acyl desaturase.

6. The genetically modified plant of claim 5, wherein the fatty acyl desaturase is CpCPRQ.

7. The genetically modified plant of claim 1, wherein the plant is Camelina sativa.

8. The genetically modified plant of claim 1, wherein the at least one insect pheromone precursor is E8,E10-12:acid.

9. The genetically modified plant of claim 1, wherein the heterologous gene is expressed in seeds of the plant.

10. The genetically modified plant of claim 1, wherein the heterologous gene further includes at least one promoter.

11. The genetically modified plant of claim 10, wherein the promoter is napin.

12. A method of producing insect pheromone precursors, said method comprising:
a) incorporating into the genome of a genetically modified plant a heterologous gene encoding at least one silencing suppressor protein and at least one enzyme selected from the group consisting of a fatty acyl desaturase, a fatty acyl elongase, a fatty acyl reductase, and an acyl-CoA oxidase;
b) breed the genetically modified plant; and
c) extract the insect pheromone precursors from the genetically modified plant.

13. The method of claim 12, wherein the at least one silencing suppressor protein is P19.

14. The method of claim 12, wherein the heterologous gene is expressed in seeds of the genetically modified plant.

15. The method of claim 12, wherein the genetically modified plant is Camelina sativa.

16. The method of claim 12, wherein the heterologous gene includes multiple copies of the same enzyme.

17. The method of claim 12, wherein the at least one enzyme is CpCPRQ.

18. The method of claim 12, wherein the heterologous gene further includes at least one promoter.

19. The method of claim 18, wherein the at least one promoter is napin.

20. A method of disrupting mating of a crop pest, said method comprising:
a) incorporating into the genome of a genetically modified plant a heterologous gene encoding a crop pest pheromone attractant, wherein the genetically modified plant releases the crop pest pheromone attractant; and
b) planting the genetically modified plant near a crop to be protected from the crop pest
